(19) United States
(12) Patent Application Publication
(10) Pub. No.: US 2025/0194976 A1
(43) Pub. Date: Jun. 19, 2025

(54) ACCELERATED ERGONOMIC COLLECTION OF CAPILLARY BLOOD

(71) Applicant: RedDrop Dx, Inc., Fort Collins, CO (US)

(72) Inventors: Kristopher Scott Buchanan, Fort Collins, CO (US); William Kristopher Buchanan, Fort Collins, CO (US); Dirk van den Boom, Encinitas, CA (US); Thomas Michael Johannsen, Centennial, CO (US); Wesley Joseph Hager, Fort Collins, CO (US); Matthew Bryan Huntingdon, Fort Collins, CO (US); Christopher Andrew Frankson, Fort Collins, CO (US); Landon Bruggeman, Fort Collins, CO (US); Dominic Castillo, Fort Collins, CO (US)

(21) Appl. No.: 19/059,103

(22) Filed: Feb. 20, 2025

Related U.S. Application Data
(63) Continuation of application No. PCT/US2023/030843, filed on Aug. 22, 2023.
Application No. PCT/US2023/030843 is a continuation-in-part of application No. 18/110,566, filed on Feb. 16, 2023, now Pat. No. 11,957,465.
Application No. 18/110,566 is a continuation-in-part of application No. 18/081,475, filed on Dec. 14, 2022, now abandoned.
(60) Provisional application No. 63/400,277, filed on Aug. 23, 2022.

(51) Int. Cl. A61B 5/15; A61B 5/151

(52) U.S. Cl. CPC A61B 5/150755; A61B 5/150022; A61B 5/150099; A61B 5/150236; A61B 5/150259; A61B 5/15149

(57) ABSTRACT

A system for accelerated ergonomic collection of capillary blood includes an apparatus and method. An apparatus includes a blood collector module with a proximal collector portion featuring a dynamic modular depressurization chamber, a depressurization piston, a precompressed volume expansion spring, and a slide latch for actuation. A distal collector portion includes a blood extraction chamber for collecting blood and plasma from the skin. A mid collector portion facilitates secure handling and includes a pressure distribution channel and a transfer module. The apparatus features a sealing surface for stable attachment and a lancet carrier with linearly arranged lancet strips. An angled collector transfer port is positioned distally. Additionally, a plasma separator module with a blood input port, plasma output port, and separator body portion channels blood through multilayer plasma separation units. A method involves firing lancets, distributing negative pressure, and regulating pressure to enhance capillary blood collection and plasma separation.

### CROSS-REFERENCES TO RELATED APPLICATIONS

The present application is a Continuation of and claims the benefit of PCT Application No. PCT/US23/30843 titled “Accelerated Ergonomic Collection Of Capillary Blood” and filed Aug. 22, 2023. PCT/US23/30843 is a continuation-in-part of and claims the benefit of U.S. application Ser. No. 18/110,566 titled “Accelerated Ergonomic Collection Of Capillary Blood” and filed Feb. 16, 2023 issued as U.S. Pat. No. 11,957,465 on Apr. 16, 2024. U.S. application Ser. No. 18/110,566 is a continuation-in-part of U.S. application Ser. No. 18/081,475 titled “Accelerated Ergonomic Collection Of Capillary Blood” and filed on Dec. 14, 2022 which claims the benefit of U.S. Provisional Application No. 63/400,277 titled “Modular Self-Collecting and Processing of Peripheral Blood” filed on Aug. 23, 2022. The disclosures of each of the foregoing are incorporated herein in their entirety to the extent permissible under applicable patent laws and rules for the relevant jurisdiction.

### FIELD

The subject matter disclosed herein relates generally to devices for blood sample collection and more particularly relates to apparatuses, systems, and methods for accelerated ergonomic collection of capillary blood and plasma.

### BACKGROUND

In today's growing market for health and wellness, blood samples and their components, such as blood plasma, are increasingly being used for diagnosis or research. Blood and plasma collection and processing can be accomplished by various conventional methods and apparatuses. However, while these methods and apparatus have been used for many years, substantial problems concerning the planning, procedural efficiency, duration and desirable amounts, purity, blood cell viability or quantities of blood cells, and the like, remain unresolved. Others can require a separate process for testing which may introduce a possibility for contamination or may require the use of trained medical professionals.

### BRIEF SUMMARY

One or more technologies are disclosed for accelerated ergonomic collection of capillary blood using the structures and/or functions of the disclosed systems and apparatuses.

In various aspects, the techniques described herein relate to an apparatus including: a blood collector module including: a proximal collector portion including a dynamic modular depressurization chamber, a depressurization piston, a precompressed volume expansion spring, and a slide latch for actuation; a distal collector portion including a blood extraction chamber formed in a collector base and configured to collect blood and plasma therein from a collection site in skin of a subject; a mid collector portion formed in a collector enclosure between the proximal collector portion and the distal collector portion of the collector enclosure to facilitate secure holding of the blood collector module between two digits of a hand, the mid collector portion further including a pressure distribution channel formed in the collector base and configured to distribute negative pressure generated by the dynamic modular depressurization chamber to the blood extraction chamber and to a transfer module coupled to the blood collector module; a sealing surface at a bottom portion of the blood collector module and is configured to stably seal the blood collector module to the skin around the collection site; the slide latch configured to be actuated by a lengthwise sliding motion of a single digit of the hand relative to the blood collector module; and a lancet carrier disposed within the distal collector portion and including one or more linearly arranged lancets strips including from three to six lancets per strip wherein in response to the lengthwise sliding motion of the slide latch, the lancet carrier fires the one or more linearly arranged lancet strips to momentarily puncture and retract from one or more rows of blood extraction slits in the collection site, wherein, in response to the one or more rows of the blood extraction slits being produced, blood is guided to flow from an opening in the sealing surface through a non-microfluidic blood flow channel for further processing; and an angled collector transfer port disposed distally of the blood collector module and configured to cause the transfer module when coupled to be angled away from the subject at an acute angle of between 10 and 45 degrees relative to the sealing surface.

In certain aspects, the techniques described herein relate to an apparatus including: a plasma separator module including: a blood input port that at least partially defines an upstream separator flow path and couples to a non-microfluidic blood flow channel of a blood collector module; a plasma output port that at least partially defines a downstream separator flow path and is configured to transfer plasma to a sample tube with a predetermined outside diameter; a separator body portion that links the blood input port and the plasma output port, the separator body portion channels blood from the upstream separator flow path to an upstream entry surface of one or more multilayer plasma separation units, wherein the separator body portion further includes the downstream separator flow path that fluidically couples a downstream exit surface of the one or more multilayer plasma separation units to the plasma output port for performing dynamic modular depressurization of the upstream separator flow path and the downstream separator flow path; and a dynamic modular depressurization regulator that, in response to exposure of the upstream separator flow path to a local atmospheric pressure (P₀) after it has been depressurized to a first negative pressure, reduces risk of hemolysis as plasma is separated through the one or more multilayer plasma separation units by regulating depressurization of the downstream separator flow path to a second negative pressure that does not go more than a predetermined limit below the P₀.

In one or more aspects, the techniques described herein relate to a method including: firing one or more lancets to produce one or more blood extraction slits in response to a lengthwise sliding motion of a slide latch on a blood collector module sealingly coupled to a collection site on a body part of a subject; distributing a first negative pressure to facilitate a flow of blood from the collection site to an upstream entry surface of one or more multilayer plasma separation units of a plasma separator module; and in response to a collection opening of the blood collector module being exposed to a current local atmospheric pressure (P₀) after a decoupling of the blood collector module from the collection site, regulating a downstream separator flow path of the plasma separator module to a second negative pressure that is closer to P₀ than the first negative pressure to limit force exerted on blood as it is separated by passing through the one or more multilayer plasma separation units.

In various aspects, the techniques described herein relate to an apparatus including: a plasma separator module including: a blood input port that at least partially defines an upstream separator flow path and is configured to couple to a transfer port of a blood collector module; a plasma output port that at least partially defines a downstream separator flow path and is configured to transfer plasma to a sample tube with a predetermined outside diameter when the sample tube is coupled to the plasma output port; a separator body portion that links the blood input port and the plasma output port, the separator body portion channels blood from the upstream separator flow path to an upstream entry surface of one or more multilayer plasma separation units, wherein the separator body portion further includes the downstream separator flow path that fluidically couples a downstream exit surface of the one or more multilayer plasma separation units to the plasma output port, wherein when the blood input port of the plasma separator module is coupled to the blood collector module and the sample tube is coupled to the plasma output port: an actuation of the blood collector module begins a collection of plasma by causing blood to flow from a collection site on skin of a subject through the upstream separator flow path to the upstream entry surface of a plasma separation membrane of each of the one or more multilayer plasma separation units; and the collection of the plasma is completed by the plasma separation module using the one or more multilayer plasma separation units to perform plasma separation on the blood collected by the blood collector module and outputting separated plasma to the sample tube when the blood collector module is decoupled from the collection site.

In certain aspects, the techniques described herein relate to an apparatus for a blood collection device to including: a lancet strip assembly including a plurality of linearly-arranged lancet strips, each linearly-arranged lancet strip including from 3 to 10 evenly distributed flat lancets aligned to configured to produce multiple rows of blood extraction slits in a collection site in skin of a subject; one or more channels between the plurality of linearly-arranged lancet strips; wherein in response to actuation, a lancet carrier propels the plurality of linearly-arranged lancet strips towards the collection site to produce the multiple rows of blood extraction slits in the skin of the subject.

### BRIEF DESCRIPTION OF THE DRAWINGS

A more particular description of the examples briefly described will be rendered by reference to specific implementations that are illustrated in the appended drawings. Understanding that these drawings depict only some examples and are not therefore to be considered to be limiting of scope, the examples will be described and explained with additional specificity and detail through the use of the accompanying drawings, in which:

FIG. 1 is a schematic block diagram illustrating an overview of a system for efficient and easy-to-use self-collecting and processing of capillary blood and/or plasma, according to one or more examples of the disclosure;

FIG. 2A is an isometric bottom view of an apparatus including a blood collector module with skin adhesive for accelerated ergonomic collection of capillary blood according to one or more examples of the disclosure;

FIG. 2B is an isometric top view of an apparatus including a blood collector module shown before slide latch actuation for accelerated ergonomic collection of capillary blood, according to one or more examples of the disclosure;

FIG. 2C is an isometric top view of an apparatus including a blood collector module shown after slide latch actuation for accelerated ergonomic collection of capillary blood, according to one or more examples of the disclosure;

FIG. 2D is a sectional view of an apparatus including a blood collector module for accelerated ergonomic collection of capillary blood before slide latch actuation according to one or more examples of the disclosure;

FIG. 2E is a sectional view of an apparatus including a blood collector module for accelerated ergonomic collection of capillary blood after partial slide latch actuation according to one or more examples of the disclosure;

FIG. 2F is an isometric bottom view of an apparatus including a blood collector module for accelerated ergonomic collection of capillary blood after full slide latch actuation according to one or more examples of the disclosure;

FIG. 2G is an enlarged inset showing a detailed view of a blood flow initiator strip in the blood collector module for accelerated ergonomic collection of capillary blood after full slide latch actuation according to one or more examples of the disclosure;

FIG. 2H is an exploded isometric side-top view of an apparatus including a blood collector module for accelerated ergonomic collection of capillary blood after slide actuation according to one or more examples of the disclosure;

FIG. 2I is an exploded isometric side-bottom view of an apparatus including a blood collector module for accelerated ergonomic collection of capillary blood after slide actuation according to one or more examples of the disclosure;

FIG. 2J is a schematic diagram of an apparatus including a blood collector module with a magnified inset view of linearly-arranged lancet strips and an angled transfer module being used for accelerated ergonomic self-collection of capillary blood, according to one or more examples of the disclosure;

FIG. 2K is an isometric view of a plurality of linearly-arranged lancet strips configured to allow blood to flow from a plurality of rows of blood extraction slits through one or more channels between the linearly-arranged lancet strips, according to one or more examples of the disclosure;

FIG. 2L is an illustration of a top view, side view, and front view, of a lancet, such as used in the plurality of linearly-arranged lancet strips;

FIG. 2M is an illustration of a top view, side view, and front view, of an enhanced edge lancet, such as used in the plurality of linearly-arranged lancet strips;

FIG. 2N is a chart comparing blood collection volumes for different lancet types;

FIG. 2O is an illustration of an apparatus including a tracking module that provides onscreen communication for a collection site preparation step for performing accelerated ergonomic collection of capillary blood, according to one or more examples of the disclosure;

FIG. 2P is an illustration of a system and method that include a blood collector module preparation step for performing accelerated ergonomic collection of capillary blood, according to one or more examples of the disclosure;

FIG. 2Q is a chart comparing blood collection volumes for different collection site preparation types;

FIG. 3A is an isometric top view of an apparatus for accelerating efficient collection of plasma for transfer in liquid form using dynamic modular depressurization that includes a blood collector module shown before actuation, a plasma separator module, and a transfer module, according to one or more examples of the disclosure;

FIG. 3B is an isometric top view of an apparatus for accelerating efficient collection of plasma for transfer in liquid form using dynamic modular depressurization that includes a blood collector module shown after actuation, a plasma separator module, and a transfer module, according to one or more examples of the disclosure;

FIG. 3C is an isometric bottom view of an apparatus for accelerating efficient collection of plasma for transfer in liquid form using dynamic modular depressurization that includes a blood collector module decoupled from a collection site, a plasma separator module, and a transfer module with collected plasma for transfer in liquid form, according to one or more examples of the disclosure;

FIG. 3D is a cross-sectional view of an apparatus for accelerating efficient collection of plasma for transfer in liquid form depicting a stage of dynamic modular depressurization in which upon actuation, air flows upstream to reach predetermined modular pressures according to one or more examples of the disclosure;

FIG. 3E is a cross-sectional view of an apparatus for accelerating efficient collection of plasma for transfer in liquid form depicting a stage of dynamic modular depressurization in which, a first modular negative air pressure accelerates the efficient flow of whole blood from a collection site to a plasma separation membrane, according to one or more examples of the disclosure;

FIG. 3F is a cross-sectional view of an apparatus for accelerating efficient collection of plasma for transfer in liquid form depicting a first stage of dynamic modular depressurization in which, a second modular negative air pressure is regulated to not go more than a predetermined limit below atmospheric pressure, according to one or more examples of the disclosure;

FIG. 3G is a cross-sectional view of an apparatus for accelerating efficient collection of plasma for transfer in liquid form depicting a stage of dynamic modular depressurization in which, the second modular negative air pressure accelerates the efficient flow of plasma but not blood cells through the plasma separation membrane for collection in the transfer module, according to one or more examples of the disclosure;

FIG. 3H is a cross-sectional view of an apparatus with a plasma separator module and an enlarged inset view depicting selected layers and structures, according to one or more examples of the disclosure;

FIG. 3I is an exploded isometric view of an apparatus with a plasma separator module for accelerating the efficient collection of plasma for transfer in liquid form depicting upstream airflow and downstream liquid flow through selected layers and structures, according to one or more examples of the disclosure;

FIG. 4A is a cross-sectional view of an apparatus with a plasma separator module integrally coupled with a blood collector module and an enlarged inset view depicting selected layers and structures, according to one or more examples of the disclosure;

FIG. 4B is an exploded isometric view of an apparatus with a plasma separator module integrally coupled with a blood collector module for accelerating efficient collection of plasma for transfer in liquid form depicting upstream airflow and downstream liquid flow through selected layers and structures, according to one or more examples of the disclosure;

FIG. 5 is a schematic block diagram of a portable electronic device for providing communications related to the use of the system for accelerated ergonomic collection of capillary blood, according to one or more examples of the disclosure;

FIG. 6 is an illustration of onscreen communications related to confirming the proper use of the system for accelerated ergonomic collection of capillary blood, according to one or more examples of the disclosure;

FIG. 7 depicts various onscreen communications associated with preparation, actuation, confirmation, and sample transportation steps for accelerated ergonomic collection of capillary blood, according to one or more examples of the disclosure;

FIG. 8 is a schematic flow chart diagram of a method of accelerated ergonomic collection of capillary blood and plasma, according to one or more examples of the disclosure; and

FIG. 9 is a schematic flow chart diagram of a method of performing plasma separation, according to one or more examples of the disclosure.

### DETAILED DESCRIPTION

As will be appreciated by one skilled in the art, aspects of the examples may be implemented as a system, apparatus, and or method.

Furthermore, the described features, structures, or characteristics of the embodiments may be combined in any suitable manner. One skilled in the relevant art will recognize, however, that embodiments may be practiced without one or more of the specific details, or with other methods, components, materials, and so forth. In other instances, well-known structures, materials, or operations are not shown or described in detail to avoid obscuring aspects of an example implementation.

As will be appreciated by one skilled in the art, aspects of the disclosure may be implemented as a system, apparatus, method, or program product. Accordingly, aspects or implementations may take the form of an entirely hardware implementation, an entirely software implementation (including firmware, resident software, micro-code, etc.), or an implementation combining software and hardware aspects that may all generally be referred to herein as a “module,” “controller,” or “system.” Furthermore, aspects of the disclosed subject matter may take the form of a program product implemented in one or more computer readable storage devices storing machine-readable code, computer readable code, and/or program code, referred to hereafter as code. The storage devices may be tangible, non-transitory, and/or non-transmission. The storage devices may not embody signals. In a certain implementation, the storage devices only employ signals for accessing code.

Various of the functional units described in this specification have been labeled as modules or controllers. Certain of the modules described in the specification are primarily mechanical and/or fluidic modules. Some functions of a module or a controller may be implemented as a hardware circuit comprising semiconductors such as logic chips, transistors, or other discrete components, or conductors.

For example, one or more modules may include an NFC tag used to convey information about a blood collector module, a plasma separator module, a transfer module, and so forth. A module or controller may also be implemented in programmable hardware devices such as field-programmable gate arrays, programmable array logic, programmable logic devices, or the like.

Certain types of modules or controllers may also be implemented in part or in whole, in code and/or software for execution by various types of processors. An identified controller or module of code may, for instance, comprise one or more physical or logical blocks of executable code which may, for instance, be organized as an object, procedure, or function. Nevertheless, the executables of an identified controller or module need not be physically located together but may comprise disparate instructions stored in different locations which, when joined logically together, comprise the module and achieve the stated purpose for the controller or module.

Indeed, a controller or a module of code may be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set or may be distributed over different locations including over different computer readable storage devices. Where a controller, module, or portions thereof are implemented in software, the software portions are stored on one or more computer readable storage devices.

Any combination of one or more computer readable medium may be utilized. The computer readable medium may be a computer readable storage medium. The computer readable storage medium may be a storage device storing the code. The storage device may be, for example, but not limited to, an electronic, magnetic, optical, electromagnetic, infrared, holographic, micromechanical, or semiconductor system, measurement apparatus, or device, or any suitable combination of the foregoing.

More specific examples (a non-exhaustive list) of the storage device would include the following: an electrical connection having one or more wires, a portable computer diskette, a hard disk, a random access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, measurement apparatus, or device.

Code for carrying out operations for some implementations may be written in any combination of one or more programming languages including an object-oriented programming language such as Python, Ruby, Java, Smalltalk, C++, or the like, and conventional procedural programming languages, such as the “C” programming language, or the like, and/or machine languages such as assembly languages. The code may execute entirely on the subject's computer, partly on the subject's computer, as a stand-alone software package, partly on the subject's computer and partly on a remote computer, or entirely on the remote computer or server. In the latter scenario, the remote computer may be connected to the subject's computer through any type of network, including a local area network (LAN) or a wide area network (WAN), or the connection may be made to an external computer (for example, through the Internet using an Internet Service Provider).

Reference throughout this specification to “one example,” “one implementation,” “an example,” “an implementation,” or similar language means that a particular feature, structure, or characteristic described in connection with the example is included in at least one example. Thus, appearances of the phrases “in one example,” “in an example,” “in an implementation,” and similar language throughout this specification may, but do not necessarily, all refer to the same example or implementation, but mean “one or more but not all examples” unless expressly specified otherwise. The terms “including,” “comprising,” “having,” and variations thereof mean “including but not limited to,” unless expressly specified otherwise. An enumerated listing of items does not imply that any or all of the items are mutually exclusive unless expressly specified otherwise. The terms “a,” “an,” and “the” also refer to “one or more” unless expressly specified otherwise.

As used herein, a list with a conjunction of “and/or” includes any single item in the list or a combination of items in the list. For example, a list of A, B, and/or C includes only A, only B, only C, a combination of A and B, a combination of B and C, a combination of A and C, or a combination of A, B, and C. As used herein, a list using the terminology “one or more of” includes any single item in the list or a combination of items in the list. For example, one or more of A, B, and C includes only A, only B, only C, a combination of A and B, a combination of B and C, a combination of A and C, or a combination of A, B, and C. As used herein, a list using the terminology “one of” includes one and only one of any single item in the list. For example, “one of A, B, and C” includes only A, only B, or only C and excludes combinations of A, B, and C. As used herein, “a member selected from the group consisting of A, B, and C,” includes one and only one of A, B, or C, and excludes combinations of A, B, and C. As used herein, “a member selected from the group consisting of A, B, and C and combinations thereof” includes only A, only B, only C, a combination of A and B, a combination of B and C, a combination of A and C or a combination of A, B, and C.

Also, as used herein, the term “about” generally means within ±10%, ±5%, ±1%, or ±0.5% of a given value or range, unless otherwise clear from context.

Aspects of the examples and/or implementations are described below regarding schematic flowchart diagrams and/or schematic block diagrams of methods, apparatuses, systems, and program products according to various example implementations.

The flowchart diagrams and/or block diagrams in the Figures illustrate the architecture, functionality, and operation of possible implementations of apparatuses, systems, methods, and program products according to various examples and implementations. In this regard, each block in the flowchart diagrams and/or block diagrams may represent a module, segment, or portion of code, which includes one or more executable instructions of the code for implementing the specified logical function(s).

It should also be noted that, in some alternative implementations, the functions noted in the block may occur out of the order noted in the Figures. For example, two blocks shown in succession may be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted example. For instance, an arrow may indicate a waiting or monitoring period of unspecified duration between enumerated steps of the depicted example. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and code.

The description of elements in each figure may refer to elements of proceeding figures. Unless otherwise clear from context, like numbers refer to like elements in all figures, including alternate implementations of like elements.

### Introduction and Overview

Generally, the present disclosure describes systems, methods, and apparatus for self-collecting and processing capillary blood. More specifically, the present disclosure relates to systems and methods for gathering a blood sample, in a closed disposable module, and a method to package that blood or its components in wet or dry forms in another device or container for further processing such as testing, storage, transportation, or use in medical diagnostic, treatment or research applications. Further, the disclosure provides ways to track the location of the device and to share device data with data networks and other devices.

The inventors of the subject matter of the present disclosure have identified a need for a cost-effective and simple blood collection device that is optimized to be self-operated by a person who needs to draw a small amount of blood to use a blood or plasma sample to make a wellness, fitness, or medical determination of the person's health.

Capillary blood and/or plasma can be useful for a variety of laboratory tests such as a basic metabolic panel, bilirubin, blood urea nitrogen/creatinine ratio, complete blood count (CBC), comprehensive metabolic panel, cortisol, ferritin, glucose, immunoglobulins tests, liver function tests, osmolality, plasma clotting time tests, thyroid function test, triglycerides, to name just a few.

Without the system, apparatus, and methods disclosed herein, existing methods may have numerous drawbacks even for relatively small amounts of blood. Some direct-to-consumer kits provide lancets and sample tubes, or lancets integrated with a microcontainer for performing finger prick blood collection. Moreover, using the finger prick method, only a limited amount of blood can be drawn. The pads of fingertips have a high density of nerve endings and finger pricks can be painful even when pricking the sides.

Pain is still a problem is some existing blood collection devices ostensibly for point of care or home use. Whether such devices cause less pain than a finger prick is a question that avoid the unpleasant that for some users, the level of pain experienced is high enough to cause fear or anxiety. For example, pain is high enough to lead to anxiety in some users when collecting blood using one existing device purportedly suitable for point-of-care or home use were the device uses a single needle or lancet to puncture skin at a collection site on a body part of a subject.

By contrast, in certain implementations disclosed herein, a unique approach to puncturing skin at a collection site taken by the inventors of the subject matter disclosed herein, may include multiple rows of lancet strips and/or may include one or more lancets with innovative single bevel blade edges, both of which alone or in combination facilitate collection of blood and plasma samples that is essentially painless or at least results in pain levels that are significantly lower than existing devices and methods.

In laboratory testing of blood serum and plasma samples, hemolysis consistently emerges as a leading preanalytical error, accounting for between 40% and 70% of unsatisfactory specimens. In the majority of cases hemolysis occurs in vitro. Hemolytic blood specimens are one of the most challenging pre-analytic issues in laboratory medicine. Not only because it is the most frequent preanalytical error, but also because it is difficult to directly influence hemolysis levels by the laboratory staff. To reduce hemolysis rates, the laboratory has to train phlebotomists in pre-analytics and blood collection and motivate them to keep hemolysis levels low. The American Society for Clinical Pathology guidance is that an optimal practice benchmark is a hemolysis rate of 2% or less.

If there is a delay in separating the plasma or serum from the cellular components of the blood, cellular metabolism continues and can lead to hemolysis. A notable proportion of hemolysis in clinical specimens is due to delays in processing. The existing solution to minimize delay in laboratories is to provide procedures and training that minimize delay when plasma is separated by centrifugation. Similarly, centrifuging at too high a speed can cause rupture of the red blood cells, leading to hemolysis. Likewise, centrifuging for too long or multiple times, even at the correct speed, can also lead to hemolysis, as the red cells can be subjected to mechanical stress for an extended period. Furthermore, failure to evenly balance samples in a centrifuge produces unequal forces that can exert mechanical stress on the cells and cause hemolysis. Moreover, centrifugation at non-optimal temperatures (too hot or too cold) may predispose the red cells to hemolysis. Even existing devices and methods for collecting blood at a point of care or at home have delays in separating plasma from blood either because of multiple steps that a user must perform or because of the slowness of the devices in separating plasma.

Accordingly, the inventors of the subject matter of the disclosure have provided a solution that represents a significant improvement over existing methods by using a device that performs both blood collection and plasma separation in rapid succession with the action required on the part of the user being a simple sliding motion to actuate the blood collection and decoupling of the device from the collection site on the skin of the user so that the dynamic modular depressurization built into the apparatuses, systems and methods disclosed herein can immediately accelerate the plasma separation process with safeguards to prevent hemolysis due to overpressure.

If a capillary blood collection device collects only a limited volume of blood (e.g., less than one hundred L), there may be an insufficient volume of blood to conduct the needed tests, and doing more than one draw with the same device may increase the risk of infection, leakage, pain, etc.

Stability during operation may be an issue if the height of a blood collection device is as large or is larger than the width of the device at the base. Such devices may be more easily tipped or unintentionally dislodged creating a risk of leakage and/or possible contamination. Furthermore, it may be challenging for a subject who is collecting blood from himself or herself to directly see a fill level of a connected sample tube if a connected collection container such as a sample tube is not angled away from the skin of the subject but is instead generally parallel to the base of the blood collection device.

In various blood collection sample circumstances, considerations such as anxiety related to seeing blood being collected from one's forearm or where concerns about potential temporary wounds being more visible on an anterior forearm, the apparatuses and methods disclosed herein may be used for blood collection on a shoulder, upper arm, or other body areas. For example, for small children, some suitable body areas for collection sites may include a lower back region, or for animals, suitable body areas may include vertical regions of skin which are shaved or otherwise hairless.

The inventors of the subject matter of the present disclosure have identified a need for a cost-effective and simple blood collection device that is optimized to be self-operated by a person who needs to draw a small amount of blood to obtain a blood or plasma sample to make a wellness, fitness, or medical determination of the person's health.

The following detailed descriptions of the drawings provide various implementations and examples of apparatuses, systems, and methods that represent significant improvements over existing technologies regarding the accelerated and ergonomic collection of capillary blood and plasma.

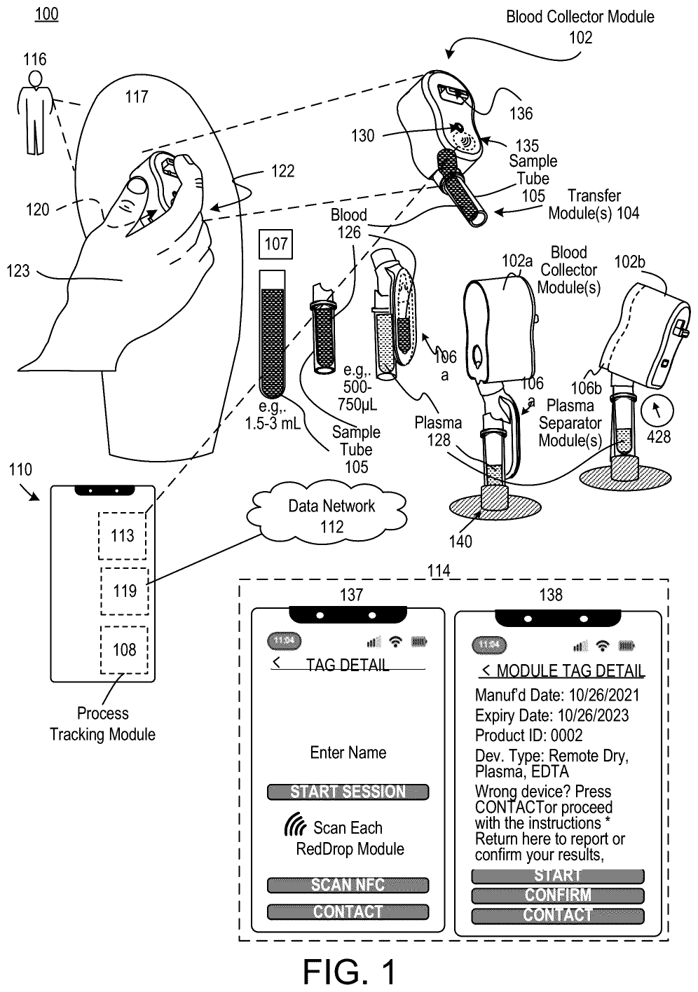

FIG. 1 is a schematic block diagram illustrating an overview of a system 100 for accelerated ergonomic collection of capillary blood, according to one or more examples of the disclosure.

Although capillary blood sampling has been used to collect very small amounts e.g., ten to twenty microliters of blood from a small puncture/prick in a finger, heel, earlobe, big toe, or palm, various problems such as the formation of calluses, low volume of blood collected, slow blood flow, and/or cumulative pain with repeated pricking.

Some capillary blood collection systems have used microneedles or microfluidics in an attempt to reduce the pain associated with finger pricks and to try to collect larger volumes of blood. However, certain systems have low-volume or slow blood flow due to the small size of the microneedles and/or microfluidic blood flow channels which may lead to incomplete blood collection or may cause hemolysis, which is the rupture, destruction, or breakdown of red blood cells which can affect laboratory results. For example, certain blood samples containing more than one hundred mg/dL of hemoglobin can cause nonspecific binding in serological tests.

Accordingly, the inventors of the subject matter disclosed herein have applied ergonomics, which is an applied science concerned with designing and arranging things people use so that the people and things interact most efficiently and safely to develop an accelerated ergonomic capillary blood collection system designed to be rapidly, safely, and easily self-operated by a subject. In certain circumstances, such as with infants, animals, and other subjects with limited physical or mental capacity, the system 100 may be operated by another person such as a healthcare professional.

The amount of blood collected using the system 100 is designed to be of a sufficient volume and quality that the blood is useful for a medical determination such as a diagnostic test. The system 100 includes three types of modules. A first type of module is a blood collector module 102. The blood collector module 102 is designed to be mechanically and adhesively coupled to a collection site of skin 122 on a body part 117 of a subject 116. In various examples, the blood collector module 102 includes an angled collector transfer port (244 that allows a transfer module 104 to be coupled an angle so that it is readily visible while gravity-assisted transfer of blood from the blood collector module 102 to the transfer module 104 is performed.

The transfer module 104 enables blood to be immediately removed from the device for immediate testing, or to be transferred to a storage location, or to a laboratory testing location. In various example implementations, certain blood components such as DNA, red blood cells, white blood cells, proteins, etc. are kept intact for further processing. It may be noted that as used herein, descriptions of blood components flowing are understood to refer to blood and its components flowing, unless otherwise clear from context. For example, in certain example implementations, the blood collector module, the plasma separator module, and various components within are designed for single use. In certain implementations, pending on the type of analyses to be conducted, both the sample and the modules may be sent to the laboratory for analysis such as for example, when collecting a plasma sample, the plasma separator module 106 may be returned to the lab so that analysis may be performed on filtered blood components remaining in the one or more multilayer plasma separation units. In other implementations, the plasma separator module and/or the blood collector module may be discarded and disposed of in a clean way, especially if done at home or in a non-medical facility.

Beneficially, the system 100 enables a subject 116 to electronically receive and/or share data related to the blood collector module 102, the plasma separator module 106, and/or the transfer modules 104, with laboratories and providers, for purposes of location tracking, safety, or device data sharing.

The system 100 may include various types of transfer modules 104, such as for example, a sample tube 105, and/or a reagent mixer module 107. Further details regarding the blood collector module 102, a plasma separator module 106, the transfer modules 104, and the process tracking module 108 are provided below.

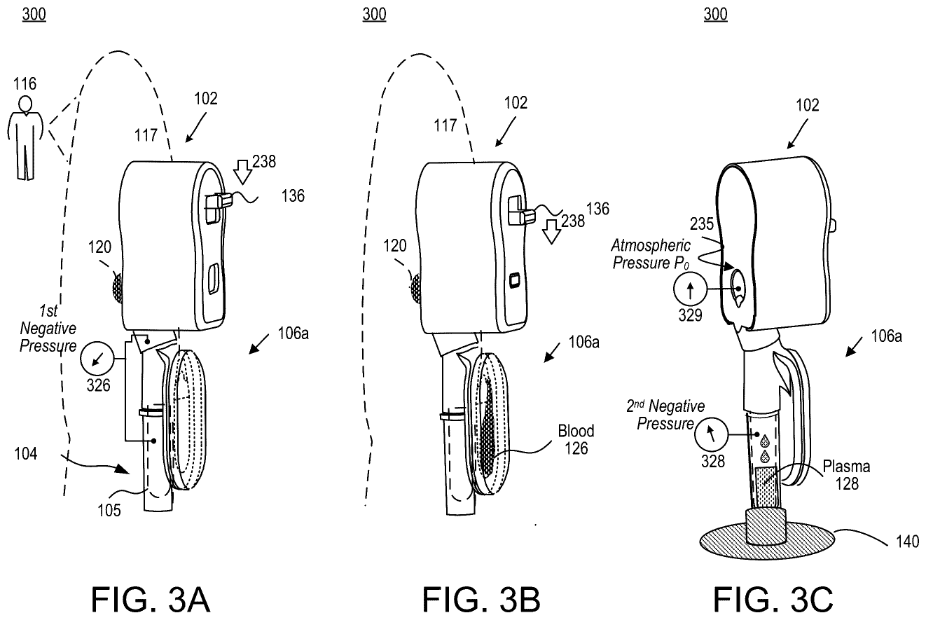

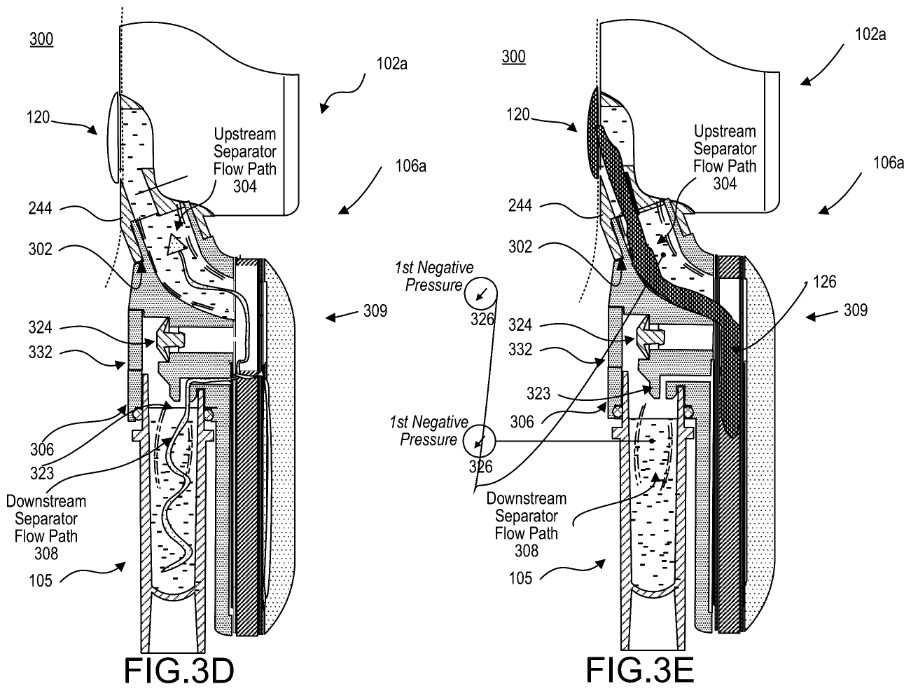

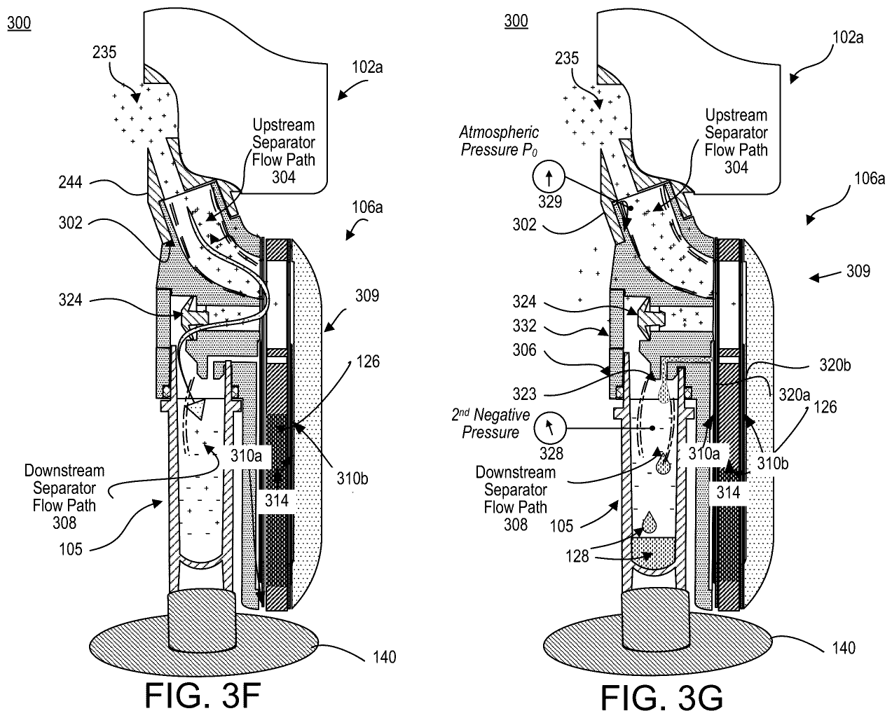

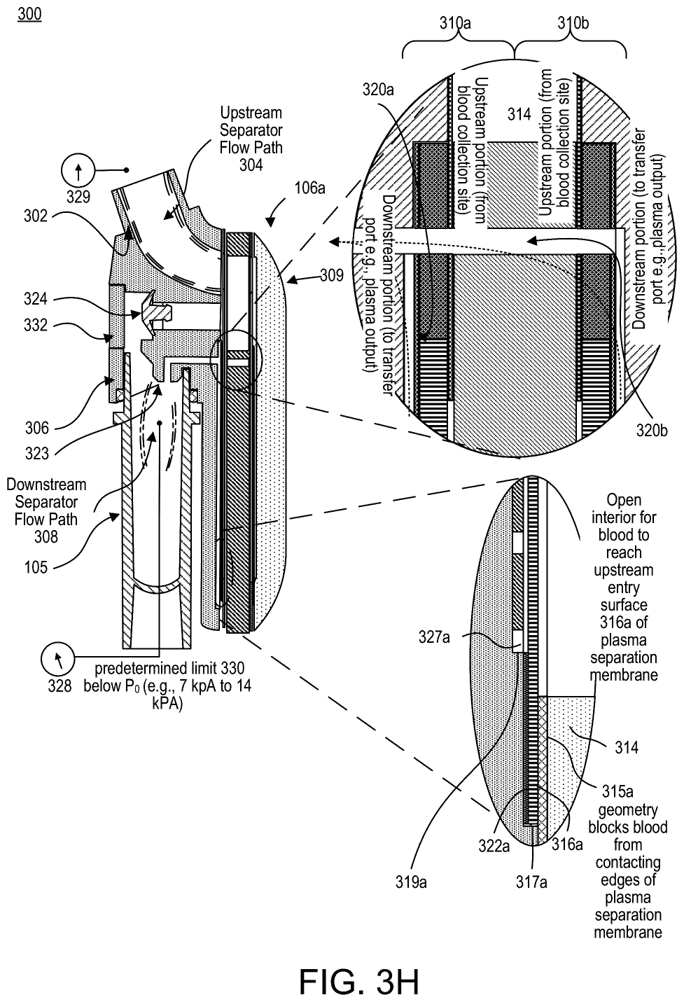

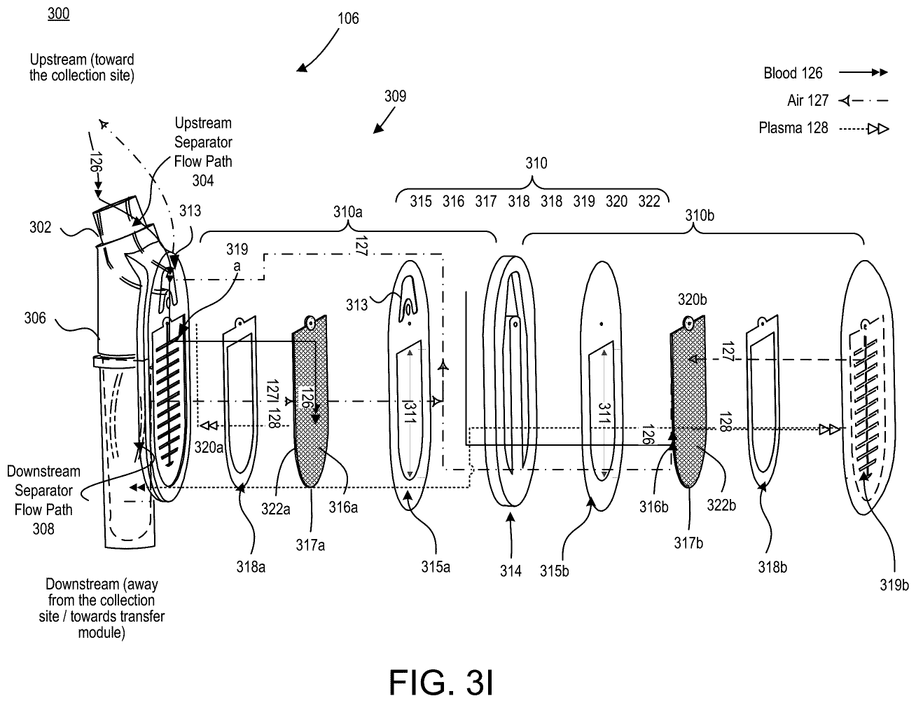

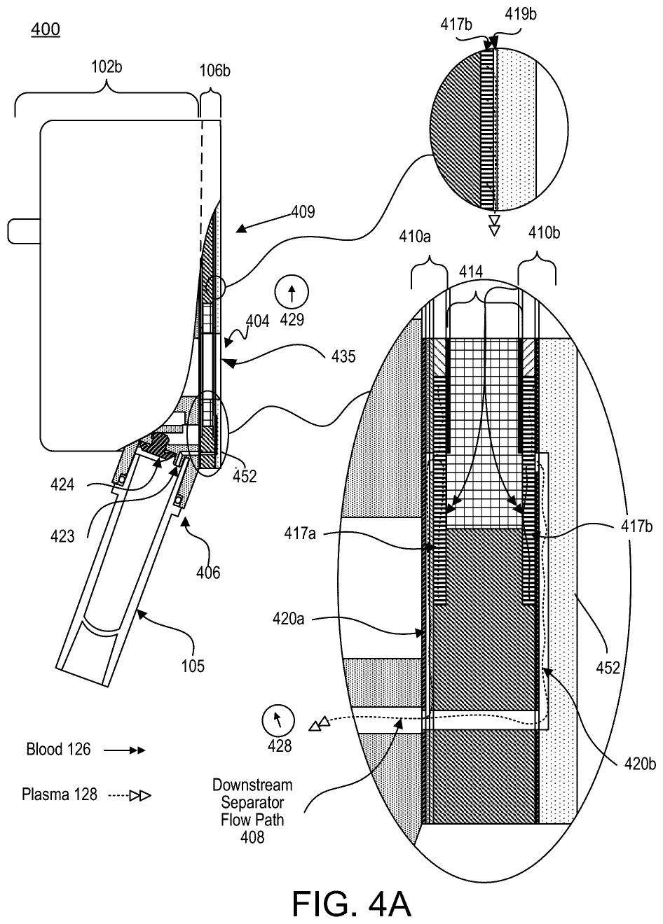

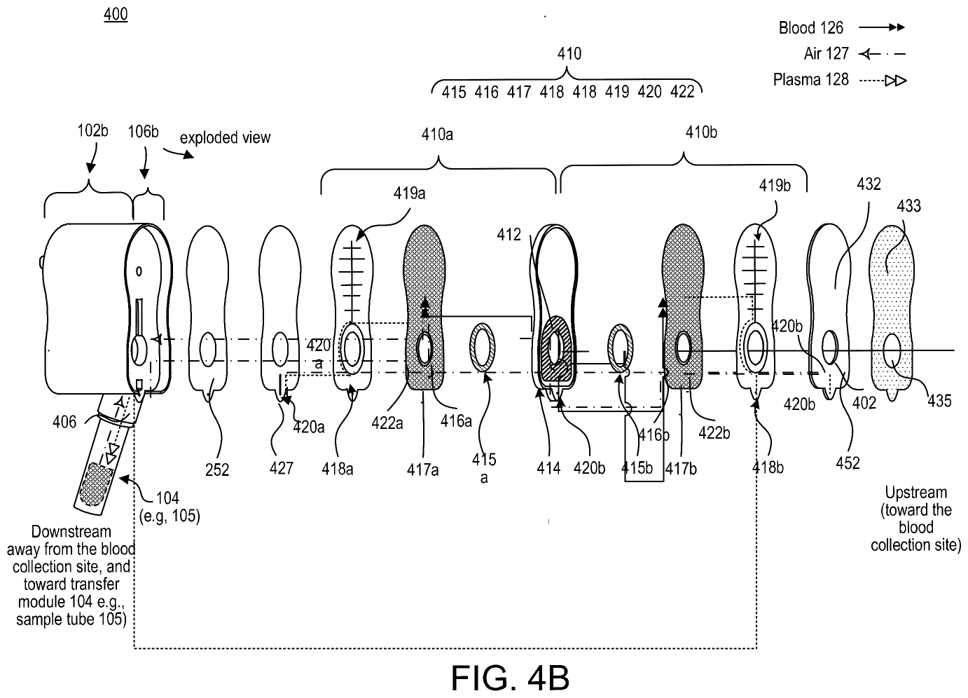

In some implementations, such as for example, the implementations of a plasma separator module 106b that is integrally coupled with a blood collector module 102a, (as described with respect to FIGS. 3A-3I with modifications further described with respect to FIGS. 4A, 4B) the plasma separator module 106b, could be referred to as a transfer module 104 in the sense that blood is transferred into the plasma separator module 106b. However, a plasma sample is transferred out of the plasma separator module 106b to another type of transfer module 104 such as a sample tube 105. In such implementations, where sample tube 105 is coupled to the collector transfer port 244 of the blood collector module 102b, one may consider the sample tube 105 as a transfer module 104 for plasma and consider the plasma separator module 106b as an integrally coupled module with the blood collector module 102b which together operate as a unitary plasma collection device. In other contexts, the term “transfer module” generally refers to separable modules used to transfer samples.

Similarly, a sample tube 105 may be used to collect plasma in various implementations and to collect blood in other implementations.

Although FIG. 1 depicts the plasma separator module 106 as being removably couplable to the blood collector module 102, in certain implementations, the blood collector module 102b and the plasma separator module 106 are integrally coupled. As used herein, “integrally coupled” means that at least a portion and the blood collector module 102 of the plasma separator module 106 are shared as a unitary structure. This approach means that the plasma separator module 106 may be included as an extended portion of the blood collector module 102 which reduces the overall size and complexity of the combination of blood collector module 102b and plasma separator module 106b is reduced.

Throughout the present disclosure, many examples and figures are depicted with the plasma separator module 106a being inseparably coupled to the blood collector module 102a. However, in most respects, unless otherwise clear from context, the structural components and functions of the plasma separator module 106b as combined with the blood collector module 102b which is integrally coupled at least in part to the blood collector module 102b are the same or similar as those of the plasma separator module 106a which is removably coupled to the blood collector module 102a. In certain limitations, the plasma separator module 106b is disposed within the same enclosure as the blood collector module 102b and has an elongated overall appearance but otherwise functions similarly. Accordingly, references and descriptions to the blood collector module 102 and the plasma separator module 106 should be understood to be fully compatible with both the integrally coupled implementation of the blood collector module 102b with the plasma separator module 106b and the separably coupled implementation of the blood collector module 102a with the plasma separator module 106a unless otherwise clear from context.

In certain implementations, a sample tube 105 may be a standardized sample tube type that has a predetermined outside diameter. Some examples of standardized sample tube based on widespread industry adoption are certain models of sample tubes available from available from Becton Dickinson such as the BD Microtainer® sample tubes which have an outside diameter of about 10 mm and the BD Vacutainer® blood collections tubes which have an outside diameter of about 13 mm. The interoperability and selection of standardized sample tubes with a predetermined outside diameter may provide beneficial technical effects, such as for example, improved and consistent dynamic depressurization because of more airtight coupling as well as fewer operational steps for analysis using instruments which are also designed to accommodate standardized sample tubes sizes.

In some implementations, sample tubes other than the Microtainer® size Vacutainer® size or may be used with similar technical effects with differing blood collection capacity. For example, standard volume of 500 μL, 750 μL, 1.5 mL, or 3 ML may be obtained as standardized sample tubes as depicted in FIG. 1 thus providing interoperability in certain instruments and same time providing increased collected specimen volume.

In certain implementations, the system 100 includes a transfer module 104 with a transfer opening at proximal end with an inner diameter of about 10 mm. In some examples, the transfer module includes a prepackaged additive selected to mix with the capillary blood to facilitate performance of one or more predetermined laboratory tests.

In certain implementations, the system 100 uses the dynamic modular depressurization to facilitate mixing of one or more reagents with collective blood and/or plasma using differences in air pressure to generate air-driven, fluid driven or mechanically driven mixing of reagents in the transfer module 104. For example, some transfer modules include one or more reagent compartments that dispense and mix reagents with the collective blood and/or plasma as it flows through one or more flow paths propelled by depressurized air within the main volume of the transfer module 104 that is lower pressure than atmospheric pressure to one or more openings on the upstream side of the reagent compartments so that the lower pressure on the downstream side of the reagent compartments causes the reagent to mix with fluids in the blood collection/plasma collection flow paths.

In various implementations, the system 100 includes a process tracking module 108 that is configured to provide information about how to perform a predetermined blood collection process using a transfer module 104 e.g., a sample tube 105, with the blood collector module 102 according to instructions determined by a healthcare provider, a requesting laboratory, an agency, an employer, or other entity involved with the selected blood testing.

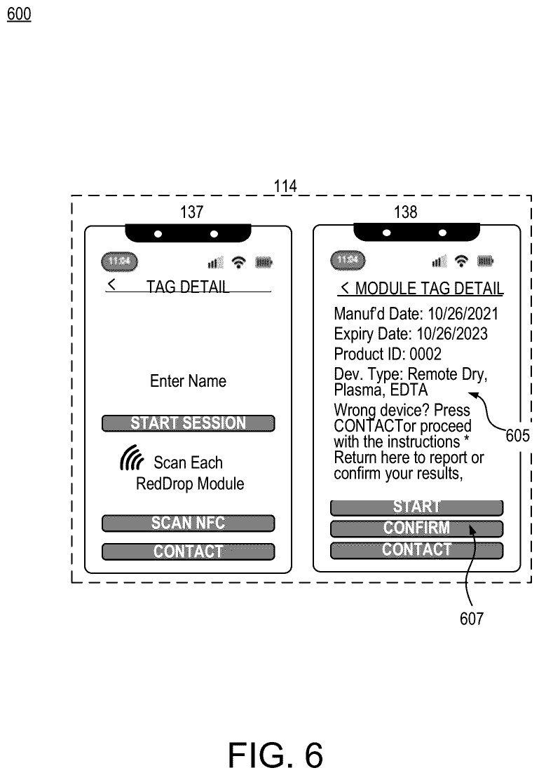

For example, as depicted in FIG. 6, a subject 116 may be instructed via an onscreen communication 137 on a display of a portable electronic device 110 such as a smart phone, to enter their name and to perform an NFC scan of each module that they intend to use. The term “NFC” refers to near-field communication which is a set of communication protocols that enables communication between two electronic devices such as the NFC scanner 113 and an NFC tag 135 over a short distance. This can be done by starting the application that implements the process tracking module 108, entering the name of the subject 116, and following the displayed instructions to scan each module using the NFC scanner 113 e.g., by touching the scan NFC icon on the application display.

When the NFC scan is completed, an onscreen communication 138 may show module information based on data bits stored in the NFC tag 135 which may also be used as an index to additional information which may be accessed through a data network 112. As depicted in the onscreen communications 137, 138, a subject 116 may be asked to enter their name, scan modules, and confirm that information displayed electronically in response to the NFC tag scan matches written or electronic information conveyed to a subject by mail or email.

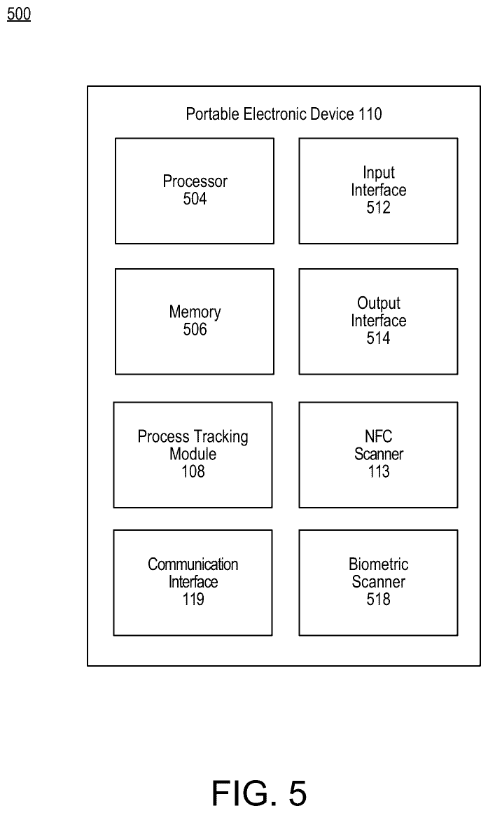

Further details about the process tracking module 108 and various content or communications that may be displayed on the portable electronic device 110 are provided below in the detailed description of FIGS. 5, 6, and 7.

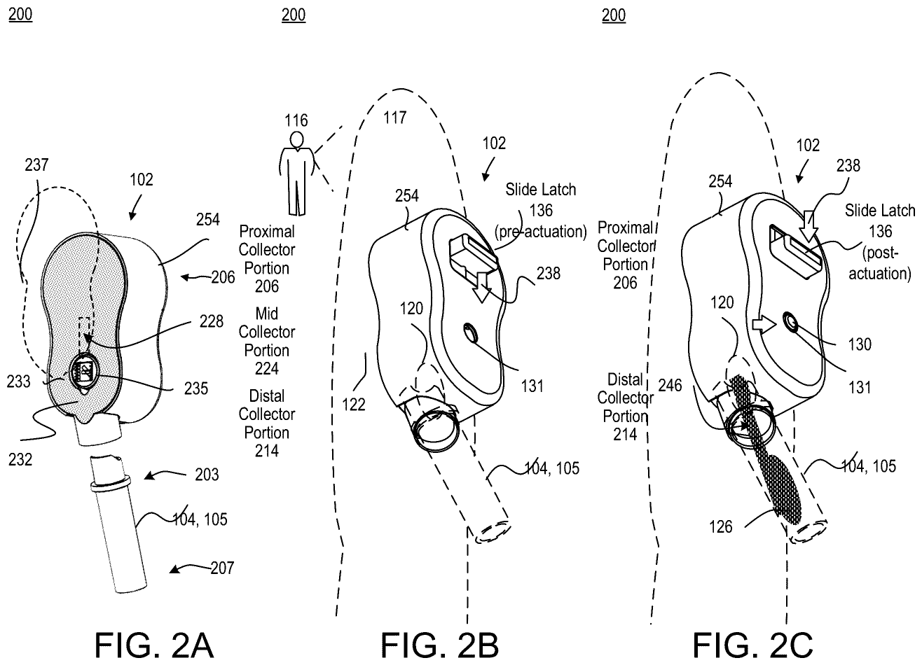

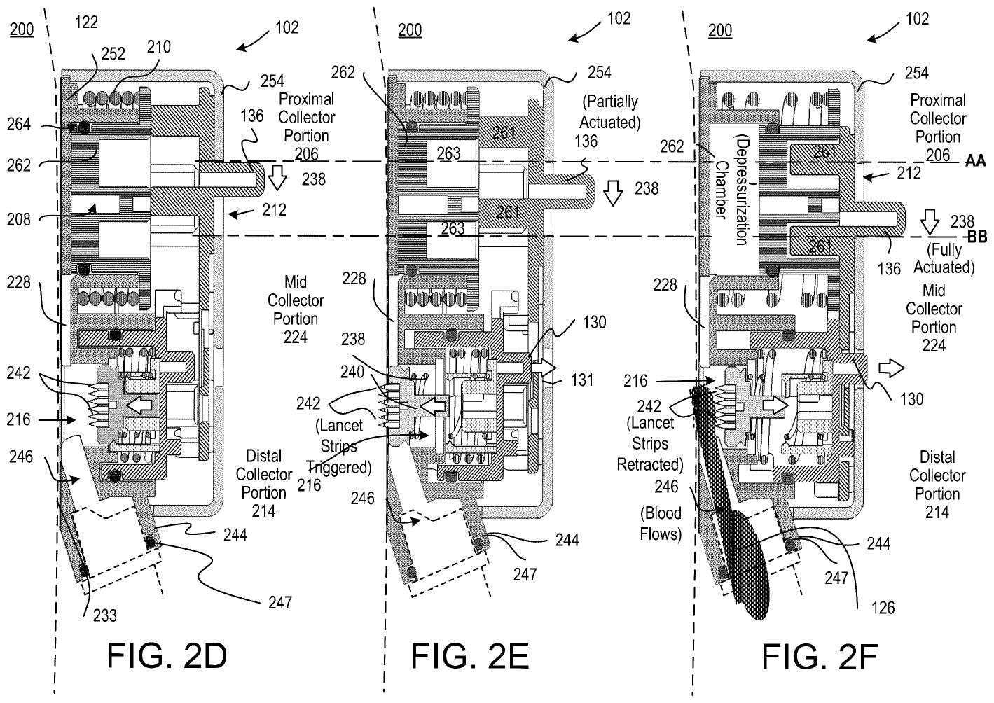

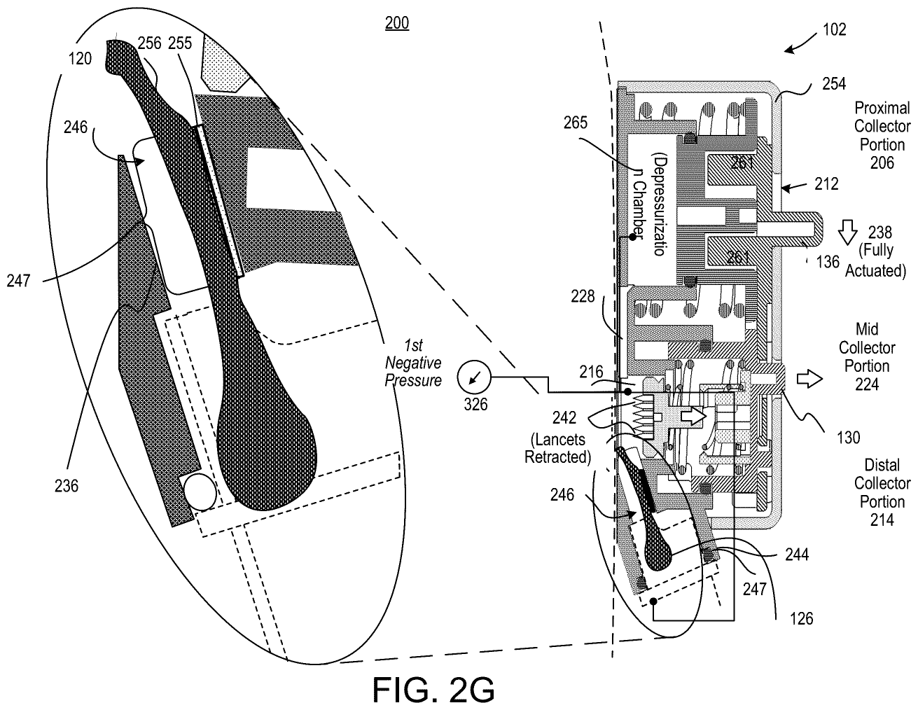

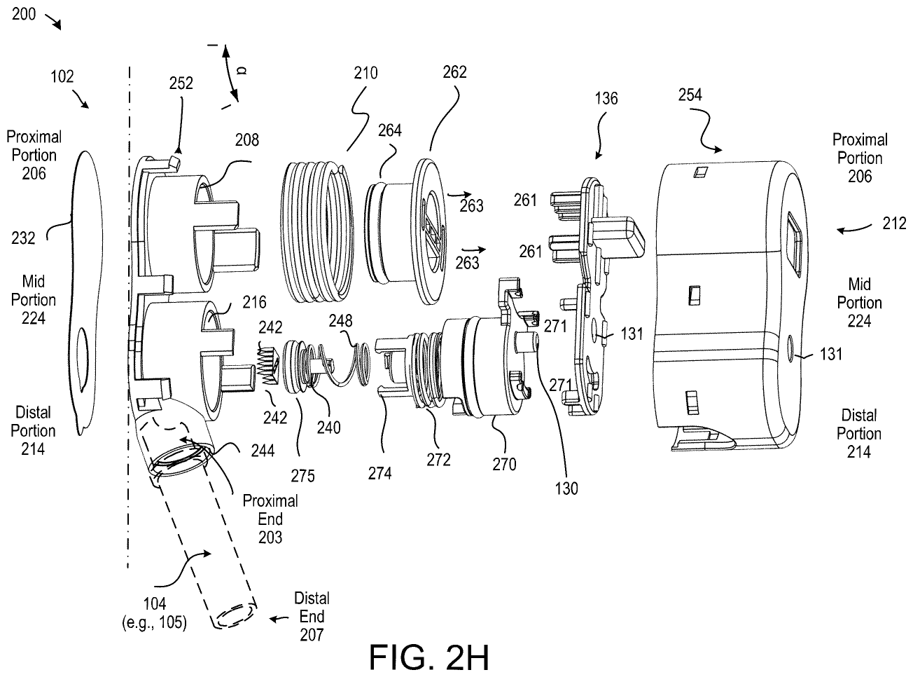

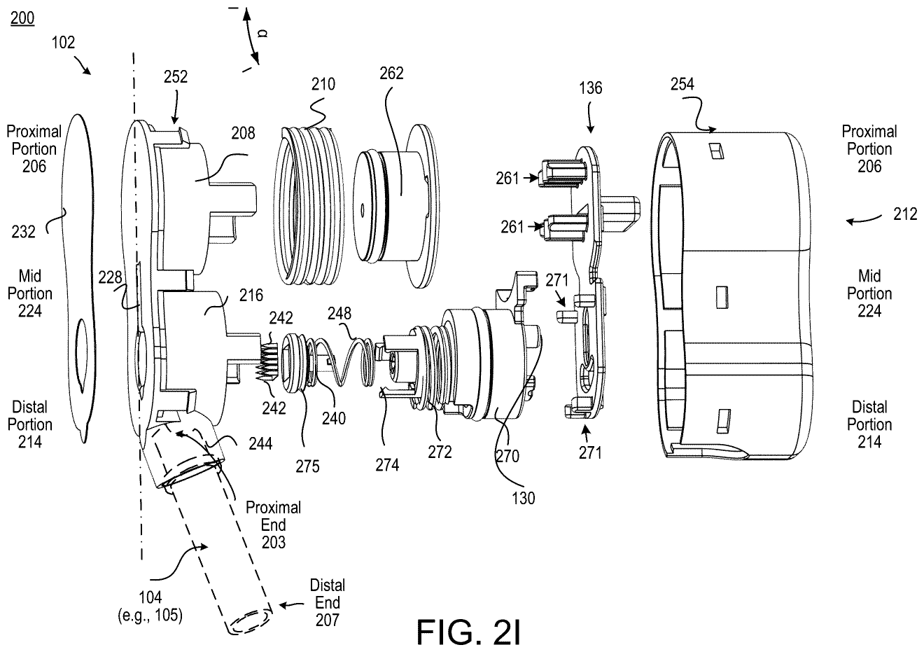

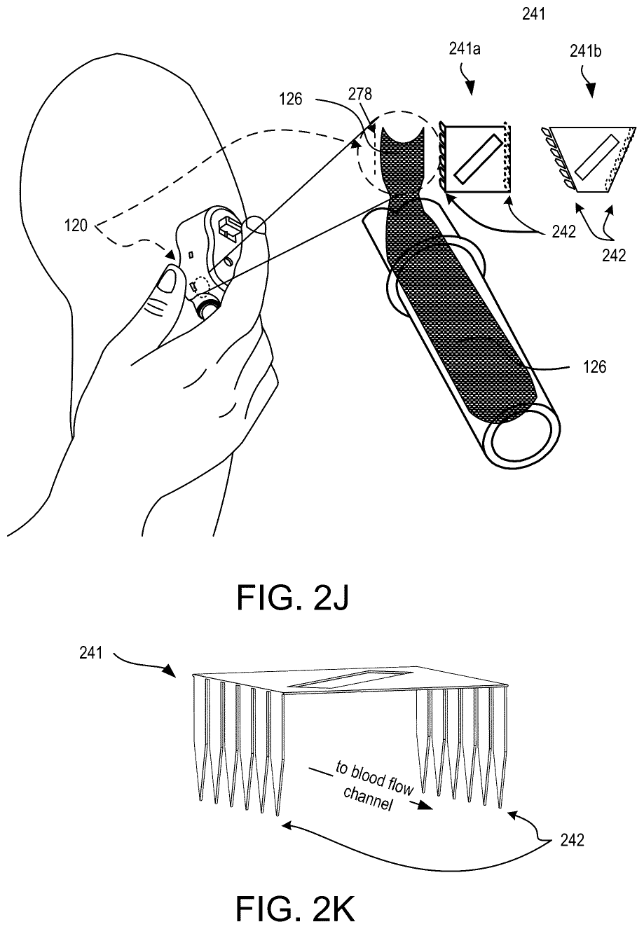

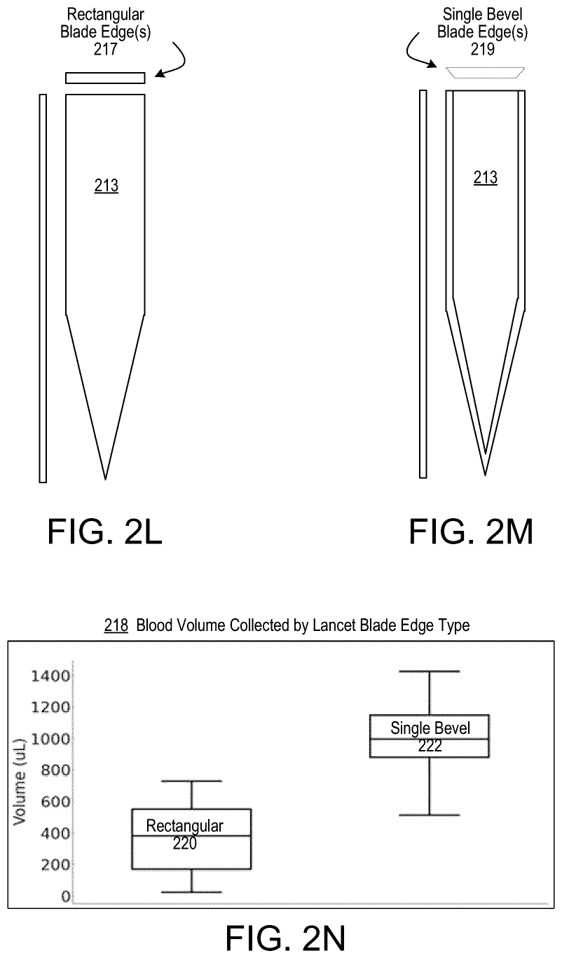

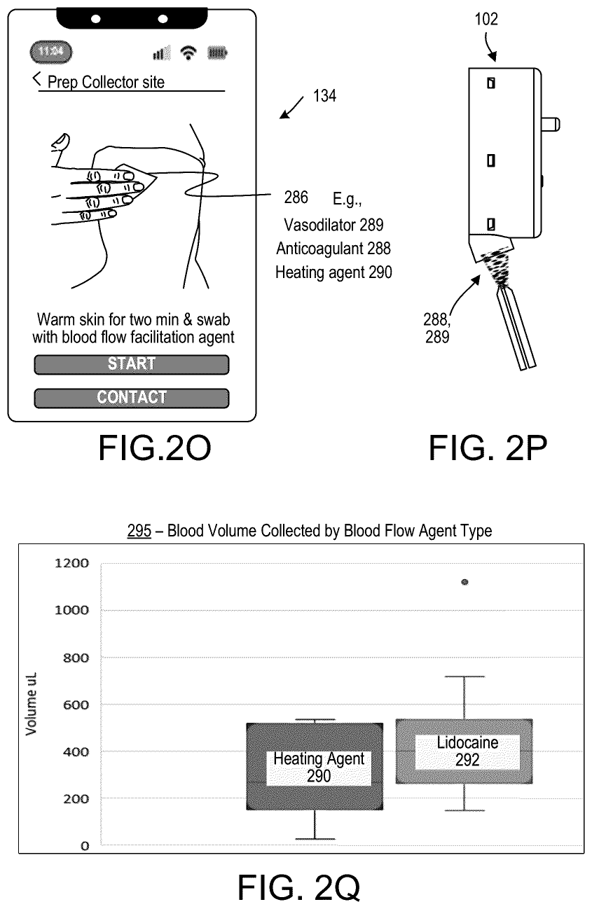

FIGS. 2A-2I. The following disclosure includes a number of different figures related primarily to the blood collector module 102, components that may be included in the blood collector module 102, internal structures, mechanisms, and functions relating to the blood collector module 102, use cases for the blood collector module 102, and so forth.

FIGS. 2A-2C illustrate and describe the high-level structures and functions of the blood collector module 102 as experienced by the subject 116 or a user if someone other than the subject 116 actuates the blood collector module 102. FIGS. 2D-2F illustrate, describe the inner components, and functions the blood collector module 102 using a cross-sectional perspective. FIGS. 2J-2K illustrate and describe details related to the blood flow speed and efficiency of blood from multiple rows of blood extraction slits created in certain implementations by multiple linearly-arranged lancet strips 242 through the non-microfluidic channel in the collector transfer port to the transfer module 104.

FIG. 2A is an isometric bottom view of an apparatus 200 including a blood collector module 102 with a skin adhesive 233 to facilitate accelerated ergonomic collection of capillary blood according to one or more examples of the disclosure.

In the selection and ergonomically beneficial skin adhesive 233 for a blood collector module 102, it is important to take into account various trade-offs relating to a weight bearing adhesive that will hold the blood collector module 102 on a body part 117 of the subject 116 for the short time it takes to collect the blood 126 and at the same time will avoid excessive skin irritation and discomfort to the subject 116 even when used frequently for the same region of skin 122. Different types of adhesives may be used for different types of subjects. Thus, safe and ergonomic removal of the blood collector module 102 is also an important factor. For example, different skin adhesives may be used for persons with skin sensitivity, infants, various types of animals, or other subjects with special requirements.

In various implementations, the skin adhesive 233 comprises a double-sided elastic adhesive film or adhesive tape that aggressively attaches to the sealing surface 232 at a subject facing portion of a collector base 252 and securely but gently adheres temporarily to the skin 122 of the subject 116.

In certain examples, the skin adhesive 233 in tape or film form may include one or more of synthetic rubber, acrylic silicone, hydrocolloid, and/or combinations thereof. In some examples, the skin adhesive in tape or film form has sufficient thickness and elasticity to elastically seal the blood collector module 102 to the skin 122 of the subject 116 with sufficient sealing quality even with subjects where there may be a few hairs or other nonuniformities in the skin 122 of the subject 116. Various examples of suitable skin adhesive 233 in tape or film form may be obtained from Vancive Medical Technologies of Chicago, Illinois, USA, or 3M Company of St. Paul, Minnesota, USA and other sources that may be known to skilled persons in the field.

In one or more examples, the blood collector module 102 is shipped with a release liner 237 which may be removed just prior to adhesively coupling the blood collector module 102 to the region of skin 122 of the subject. The skin adhesive 233 facilitates sealing the sealing surface 232 on the collector base 252 (depicted in FIGS. 2D-2F) so that depressurized volume can be formed above the collection site 120 to facilitate accelerated ergonomic blood collection.

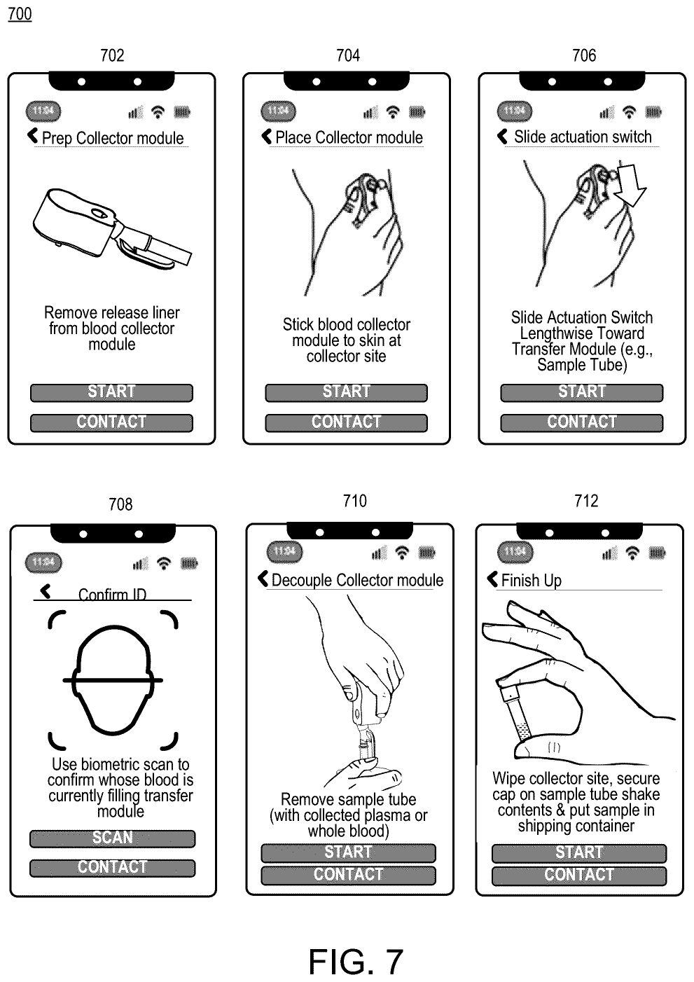

As depicted in FIG. 2A, the skin adhesive 233 in film or tape form also serves as a subject facing wall of a pressure distribution channel 228 formed in a collector base 252 where the pressure distribution channel 228 facilitates depressurization at a collection opening 235 in a distal collector portion of the collector base 252 (as further depicted and described with respect to FIG. 2G). With the blood collector module 102 position on the skin 122 of the subject 116 such that the proximal collector portion 206 is higher than the distal collector portion 214, accelerated blood flow is enhanced as shown in FIG. 2C. This improvement is further depicted a in the onscreen communication 710 shown in FIG. 7.

Another ergonomic improvement of the disclosed angled collector transfer port 244 is that it facilitates decoupling of the blood collector module 102 with less mess. In certain implementations, with the transfer module 104 being coupled to the blood collector module 102 and disposed perpendicularly to a support surface, the angled collector transfer port 244 causes the collection opening 235 in the sealing surface 232 of the blood collector module 102 to face at least partially upward to inhibit blood residue from spilling from the collection opening 235 during decoupling of the transfer module from the angled collector transfer port 244.

When blood collection is completed, the transfer module 104 may be rested perpendicularly to a surface such as a table or a desk and the blood collector module 102 may be twisted off in such a way that spillage from the collection opening 235 does not occur because holding the transfer module 104 perpendicular to the surface causes the opening to be at least partially angled upwards thus inhibiting spillage.

FIGS. 2B-2H provide further details regarding the structures and functions related to the blood collector module 102.

FIG. 2B is an isometric top view of an apparatus 200 including a blood collector module 102 shown before slide actuation for accelerated ergonomic collection of capillary blood. In many implementations, the slide latch 136 of the blood collector module 102 is configured to be easily actuated by a subject who is doing self-collection of blood, such as for example, when requested by a physician, employer, or other entity, so that one or more laboratory tests may be performed on a blood sample from the subject.

One of the various ergonomically beneficial aspects of the slide latch 136 of the blood collector module 102 is that because there is no significant counterforce pushing back against a user's finger or hand in a direction opposite the direction of actuation, actuation of the blood collector module 102 is significantly easier than existing devices as will be explained in more detail below.

Another ergonomic aspect is that there is an actuation indicator opening 131 through which a user can see or feel whether the blood collector module 102 has been actuated. In existing devices, the amount of force needed to perform actuation is significant and the pressure or pain may be significant enough that the user may find it hard to tell the blood collection device has been actuated. Further details are provided below about how the blood collector module 102 solves such problems found in existing devices.

As further depicted in FIG. 2B, with the slide latch 136 in a pre-actuation condition, the user can see a portion of the slide latch 136 through the actuation indicator opening 131. In various implementations, the color of the protruding portion of the slide latch 136 which is moved by the user using a lengthwise sliding motion 238 is the same color as a portion of the slide latch that can be seen through the actuation indicator opening 131. Prior to actuating the slide latch, a transfer module 104, such as for example, a sample tube 105 is coupled to the blood collector module 102 in a way that facilitates ergonomic coupling, viewing, and decoupling of the transfer module throughout the collection of capillary blood.

FIG. 2C is an isometric top view of an apparatus 200 including a blood collector module 102 shown after slide actuation for accelerated ergonomic collection of capillary blood. As noted with respect to FIGS. 1, 2B, in some implementations, the blood collector module 102 includes a slide latch 136 that is configured to be ergonomically actuated by a lengthwise sliding motion 238 of a single digit (e.g., index finger) of the hand 123 relative to the blood collector module 102.

Various ergonomic aspects of the blood collector module 102 are further enhanced by the accelerated blood flow facilitated by the structures and functions disclosed herein. In some existing systems, a user or a subject may need push down on an actuator for several seconds or more and may further need to wait as much as one minute before being able to tell whether they device has been properly actuated and blood is beginning to be collected. However, as described in more detail below, a user using the blood collector module 102 can determine immediately that the blood collector module 102 has been actuated by viewing or feeling the position of the slide latch 136 and by viewing or touching the actuation indicator 130, which may have a different color than the slide latch and may be felt and seen protruding from the actuator indicator opening.

As a further ergonomic improvement, in various implementations, the blood collector module 102 includes a mid collector portion 224 formed in the collector enclosure 254 between a proximal collector portion 206 and a distal collector portion 214 of the collector enclosure 254 to facilitate secure holding of the blood collector module 102 between two digits (e.g., thumb and middle finger) of a hand 123.

Furthermore, in various implementations because the blood collector module 102 includes a non-microfluidic blood flow channel 246 that is at least 30% as large as the minimum inner cross-sectional area of the transfer module 104 through which blood 126 collected using the blood collector module 102 is configured to flow, accelerated ergonomic collection of capillary blood is facilitated.

FIG. 2D is a sectional view of an apparatus 200 including a blood collector module 102 for accelerated ergonomic collection of capillary blood before slide latch actuation. Distinct from some existing blood collection devices in which create a reduced pressure volume through which the actuator and one or more needles move, the blood collector module 102 disclosed herein ergonomically separates the dynamic modular depressurization structures and functions, which are generally disposed in a proximal collector portion 206 of the blood collector module 102 from the blood extraction structures and functions including the or more more lancets that may in certain implementations be arranged as lancet strips, which are generally disposed in a distal collector portion 214 of the blood collector module 102. The actuation structures and functions including slide latch 136 are generally separate from but spanning across both the dynamic modular depressurization structures and functions in the proximal collector portion and the blood extraction structures and functions in the distal collector portion 214.

Furthermore, in some implementations, the ergonomic shape of the collector enclosure 254 of the blood collector module 102 it is enhanced by a mid collector portion 224 that is concave or has other grip facilitating features such as knurled sides, making it easier to position on the skin, easier to remove, easier to actuate, and giving it an overall much lower profile than existing devices in which various types of vacuum generation structures and functions, blood extraction structures and functions, and actuation structures and functions, would typically have a higher and/or larger profile and may be more cumbersome to utilize.

In certain implementations, enclosures with different ergonomic shapes other than concave may include planar portions, convex portions or a mixture of different shapes and textures to facilitate handling while still preserving efficient use of the mechanisms and structures disclosed herein.

FIG. 2E is a sectional view of an apparatus 200 including a blood collector module 102 for accelerated ergonomic collection of capillary blood after partial slide latch actuation. Various accelerated ergonomic capillary blood collection functions are configured to be performed based on the unique structure of the slide latch 136 interacting across both dynamic modular depressurization structures and functions as well as blood extraction structures and functions. For example, one notable improvement over existing devices is that even when using the collector base 252 and collector enclosure 254, the timing of the depressurization piston and the triggering of the lancets strips is mechanically selectable by a configuring the blood collector module 102 with a slide latch 136 that has different positioning of the depressurization piston blocking members 261 and the blocking elements 271 of the extraction collar which are shown and described in more detail with respect to FIGS. 2H and 2I.

FIG. 2F is an isometric bottom view of an apparatus 200 including a blood collector module 102 for accelerated ergonomic collection of blood after fully actuating the slide latch 136. The relative positioning of the slide latch 136 to the dynamic depressurization structures in the proximal collector portion 206 and the blood extraction structures is the distal collector portion 214 is depicted for a circumstance in which it is preferable to trigger the firing of the one or more lancets which may in some implementations be arranged as lancet strips as depicted in FIG. 2E to puncture skin at the collection site with earlier timing selected then the timing selected for dynamically depressurizing in the depressurization chamber 265.

In certain implementations, a first negative pressure 326 (described in more detail with respect to FIGS. 3D and 3E) in the blood extraction chamber 216 present in the first stage of the dynamic modular depressurization causes selected blood extraction slits 278 to widen to enhance blood flow. For example, the blood extraction slits 278 that are disposed between other blood extraction slits 278 may be widened by the first negative pressure 326. Similarly, the blood extraction slits 278 which are in a more open region of the collection site 120 may experience greater levels of stretching caused by the negative pressure which may cause widening of the blood extraction slits 278.

However, not only can the order of the skin puncturing and dynamic depressurizing functions depicted as shown, reversed, or configured to occur at the same time, but even within a particular order the timing of each function is configurable based on positioning of the depressurization piston blocking members 261 and blocking elements 271 which are depicted and described in more detail with respect to FIGS. 2H and 2I.

FIG. 2G is an enlarged inset showing a detailed view of a blood flow initiator strip 247 in the blood collector module 102 for accelerated ergonomic collection of capillary blood after full slide latch actuation according to one or more examples of the disclosure.

In some devices with a channel for blood flow, one problem that may occur is that blood may occasionally become suspended or stuck in the channel. Blood may become stuck in the channel if during its travel to its ultimate destination (e.g., a sample tube 105 for collecting the blood) a drop of the blood makes contact with all sides of the channel. Under such circumstances, surface tension forces may become great enough to form a meniscus that effectively blocks return air flow and stops the blood from moving downstream.

Another problem that may occur is that an initial volume of blood flowing from the collection site 120 may be damaged due to hemolysis which may be caused by the lancets striking the capillary blood vessels and splitting some of the red blood cells it comes in contact with. This may cause the initial volume of blood that is collected to have unacceptably high levels of hemolysis. Although dynamic modular depressurization reduces the likelihood of hemolysis related to over pressure of blood against the plasma separation membrane, the inventors of the present subject matter have developed a further approach to reduce the impact of hemolysis related to puncturing of red blood cells on blood samples and plasma samples to be tested.

Accordingly, in various implementations, the blood collector module 102 includes a blood flow initiator strip 247 adhesively coupled to an inner surface of a base-side portion of a blood flow channel wall 236 that defines non-microfluidic blood flow channel 246 near the collection opening 235. In some implementations, the blood flow initiator strip 247 comprises a plurality of layers. For example, in various implementation, the blood flow initiator strip 247 comprises a first adhesive layer 255 and an absorption layer 256.

In other implementations, the blood flow initiator strip further comprises second adhesive layer (not shown), and a hydrophilic wicking layer (not shown).

In various implementations, one side of the first adhesive layer 255 adhesively couples the blood flow initiator strip 247 to the inner surface of the blood flow channel wall 236 and an opposite side of the first adhesive layer 255 is adhesively coupled to the absorption layer 256. Not every biocompatible adhesive will adhere to biocompatible plastics and to absorption layers without potential delamination. The inventors of the subject matter disclosed herein have determined that one example of a suitable adhesive layer is R515 high-tech rubber film tape available from JACO Aerospace Products of Valencia, CA is one source of suitable material for the first adhesive layer 255 due to its ability to adhere both to the types of plastic suitable for forming the blood collector module 102, and for adhering to the other layers of the blood flow initiator strip 247.

In one or more example implementations, the absorption layer 256 comprises a cellulose fiber filter paper that is used to absorb an initial volume of blood such as for example, the first 5 uL-10 uL of blood that flows from the collection site into the non-microfluidic blood flow channel 246. Absorbing the initial volume of blood effectively saturates the absorption layer 256 and traps a significant portion of the blood that may have been highly hemolyzed. The rest of the blood flowing from the collection site passes over the saturated portion of the absorption layer 256 so that the blood and plasma collected for testing does not include the initial highly hemolyzed portion. Other suitable materials which have similar characteristics may be utilized in the absorption layer 256.

The inventors of the subject matter disclosed herein have determined that one example of a suitable material for the absorption layer 256 is Whatman® quantitative filter paper, hardened low-ash, Grade 54 due to its absorption-to-thickness ratio as well as its ability to retain the absorbed blood. This allows the blood flow initiator strip 247 to not be too thick and to still absorb enough blood to be effective, as well as be confident that the absorbed blood will not leach out from the paper and into the sample Whatman® is trademark of Cytiva, 100 Results Way, Marlborough, MA. Various materials may be utilized to form the absorption layer 256 provide that they absorb and retain similar volumes of blood.

In various implementations which include a hydrophilic wicking layer (not shown), the hydrophilic wicking layer is offset downstream relative to the absorption layer 256 thereby exposing a surface area of the absorption layer 256 as the first area to contact the initial volume of blood (e.g., to absorb the highly hemolyzed blood). In certain implementations, the hydrophilic wicking layer is extended in a downstream direction which provide additional wicking surface area and extends further into the downstream region which may facilitate flow. The inventors of the subject matter disclosed herein have determined that one example of a suitable material for the hydrophilic wicking layer is 3M 9984 is available from 3M Corporation of Saint Paul, MN. It is possible to use other materials that have corresponding qualities.

FIG. 2H is an exploded isometric side-top view of an apparatus 200 including a blood collector module 102 for accelerated ergonomic collection of capillary blood. This exploded top view highlights the ergonomic and efficient design of the blood collector module 102. Three different springs are provided including a volume expansion spring 210, a collar spring 272, and a lancet carrier spring 248 are shipped in a precompressed state. Together, and individually, these springs represent a significant ergonomic improvement over existing devices where a spring or a springy elastomeric member must be pressed upon or otherwise have a counter force applied to it to operate such devices.

The exploded top view of FIG. 2H highlights that in various implementations, the blood collector module 102 may be manufactured using a relatively small number of molded biocompatible components. Additionally, the exploded top view provides a three-dimensional perspective of the lancet carrier latch 274 utilized in the depicted implementation which retains the multiple lancet strips 242 from being fired into the skin until the timing configured by the slide latch 136 being actuated by sliding causes the precompressed lancet carrier spring 248 to propel the lancet strips 242 into the skin. In some implementations, the lancet carrier latch 274 includes three spatially distributed prongs that assist in retaining the lancet carrier securely in place until the slide latch 136 is actuated. Further details about the depicted implementation are provided below.

FIG. 2I is an exploded isometric side-bottom view of an apparatus 200 including a blood collector module 102 for accelerated ergonomic collection of capillary blood. FIG. 2H is similar to FIG. 2G except that exploded side-bottom view provides a three dimensional perspective highlighting one example implementation of the pressure distribution channel formed in the mid collector portion 224 of the collector base 252. It may be noted that although various example implementations depict the mid collector portion 224 is having a concave shape, the shape of the mid collector portion 224 may be generally rectangular, elliptical, or may have any shape that accommodates the internal components within the enclosure and still retain the overall benefits of the structures and functions disclosed herein and may include other features to facilitate ergonomic handling Accordingly, references to the mid collector portion 224 throughout the disclosure using the term “concave mid collector portion” 224 should be understood as exemplary and not limiting, unless otherwise clear from context.

With regard to FIGS. 2A-2I, it may be noted that within the detailed description, the use of the same or similar numbers referring to different components and subcomponents of the blood collector module 102 which are included in more than one of FIGS. 2A-2I should be understood to refer to the same structures and functions unless otherwise clear from context. FIGS. 2A-2I provide different types of detailed views of various example implementations of structures and functions that may be included in the blood collector module 102. Various elements or components of the blood collector module 102 are depicted using reference numbers in FIGS. 2A-2H that may be included in certain figures and may not be repeated in portions of the detailed description corresponding to the Figures in order to highlight different aspects of the blood collector module 102.

Implementation of these ergonomic structures and functions in the blood collector module 102 specifically improve the ease of operation, stability, and reliability of the blood collector module 102 disclosed herein relative to existing devices which exhibit many of the ease-of-use, reliability, and stability problems described in the Introduction and Overview section that precedes the detailed description of FIG. 1.

Similarly, accelerating features including a non-microfluidic blood flow channel 246 and an angled collector transfer port 244 disposed at the distal collector portion 214 of the blood collector module represent substantial improvements over existing technologies which exhibit various problems in slow blood flow rate, coagulation, and incomplete blood collection some of which are described in the Introduction and Overview section that precedes the detailed description of FIG. 1.

In various examples, the blood collector module 102 includes a collector base 252, and collector enclosure 254, a slide latch 136, and a skin adhesive 233 for securing the collector base 252 to the skin 122 of the subject 116. Accordingly, the blood collector module 102, in various examples, includes a proximal collector portion 206 comprising a dynamic modular depressurization chamber 208 formed in the collector base 252, a depressurization piston 262, and a precompressed volume expansion spring 210, and a slide latch slot 212 in the collector enclosure 254.

In the pre-actuation state, the depressurization piston 262 is biased by the volume expansion spring 210 in a precompressed state and is retained in a pre-actuation/pre-depressurizing position by a portion of the slide latch 136. The depressurization piston 262 includes an O-ring 264 that hermetically seals against the walls of the dynamic modular depressurization chamber 208 such that when the slide latch 136 is actuated by a lengthwise sliding motion 238, the precompressed volume expansion spring 210 is no longer retained and expands pushing a depressurization piston 262, away from the sealing surface 232 thereby depressurizing the dynamic modular depressurization chamber 208.

More specifically, in an implementation depicted in FIGS. 2E, 2F and 2G, when one or more depressurization piston blocking members 261 are slid far enough distally toward the transfer module 104 that the depressurization piston cavities 263 can be pushed away from the sealing surface so that the depressurization piston blocking members 261 are positioned within the depressurization piston cavities 263, the precompressed volume expansion spring 210 expands away from the sealing surface 232 and depressurizing the dynamic modular depressurization chamber 208 by the effective expansion of volume of air sealed within.

Similarly, a distal collector portion 214 of the slide latch 136 includes blocking elements 271 that maintain the extraction collar 270 with the collar spring in the precompressed state. When the slide latch is slid lengthwise toward the transfer module 104, far enough that the blocking elements 271 no longer press against the extraction collar 270, the extraction collar 270 will be pushed away from the sealing surface such that the lancet carrier latches 274 are pulled away from the lancet carrier foot 275 which triggers the lancet carrier 240 to be propelled toward the sealing surface 232 such that the one or more lancets 213 which may in various examples be arranged as lancet strips 242 momentarily puncture the skin 122 of the subject 116 creating rows of blood extraction slits 278 in the skin 122 of the subject 116.

It may be noted that although, many of the examples illustrated herein depict and describe one or more lancets 213 as being configured as linearly-arranged lancet strips 242, certain implementations of the apparatuses, systems and methods, described or depicted herein as having linearly-arranged lancet strips 242 may also be implemented using a single lancet that has been designed to minimize pain e.g., by having one or more particularly sharp blade edges. In some embodiments, the apparatuses, systems, and methods described herein may be implemented using one or more lancets arranged in geometries other than linearly arranged lancet strips without departing from the benefits and improvements provided by the other components and combinations of components described and depicted throughout this disclosure.

In response to the slide latch 136 being actuated by the lengthwise sliding motion 238, the actuation indicator 130 is configured to both visually and haptically indicate that the blood collector module 102 has been actuated. More specifically, in one or more implementations, the actuation indicator 130 is formed on the outward facing surface of the extraction collar 270 using material that has a different appearance e.g., different color such as red that becomes visible when the extraction collar 270 pushes outward extending the actuation indicator 130 through the actuation indicator openings 131 in the distal collector portion 214 of the slide latch 136 and the collector enclosure 254.

Accordingly, the visually different appearance of the actuation indicator 130 from the collector enclosure 254 can be seen when the slide latch 136 is in the fully actuated position depicted in FIGS. 1, 2C, and 2F. Likewise, because the actuation indicator 130 protrudes slightly above the outward surface of the collector enclosure 254 at the actuation indicator opening 131, a person can use the sense of touch to feel the protruding actuation indicator 130 thereby confirming that the slide latch 136 has been fully actuated.

This actuation indicator 130 is an ergonomic improvement over existing blood collection devices when used may create concern or confusion for a user because it is challenging to determine whether such existing blood collection devices have indeed been activated.

Furthermore, the disclosed approach to dynamic modular depressurization solves several problems found in existing blood collection devices. One problem with vacuum-assisted blood collection is found in existing designs for blood collection devices which use a first approach for vacuum generation of incorporating into the blood collection device, a self-contained vacuum chamber, or a vacuum chamber having a volume that is at a pressure less than ambient pressure prior to actuation. However, to maintain a pressure less than ambient pressure prior to actuation for any substantial period of time suggests that such a pre-depressurized chamber should either be nearly leakproof or potentially risk failure upon actuation because the pressure differential between the vacuum chamber and ambient pressure is less than desired due to leakage over time.

Accordingly, in view of the challenges associated with maintaining less than ambient air pressure over time in a blood collection device vacuum chamber, a second approach taken in existing designs is to push, depress, or squeeze an elastomeric component such as a bulb, bellows, flexible concave membrane, or similar vacuum source that can be compressed by a user such that air is expelled and when the pushing or squeezing stops, a partial vacuum is generated by the return of the elastomeric component to its original shape. Unfortunately, this second approach may also present several problems.

For example, to evacuate air out of an elastomeric vacuum generating component by pushing, depressing, squeezing or the like, may require significant effort on the part of the person doing the pushing, depressing, squeezing, etc. Some subjects who need or want to have blood tested fairly regularly may lack the strength to push, depress, squeeze the elastomeric component (or similar) completely so that the level of air pressure reduction may be inconsistent from time to time which may result in incomplete blood collection e.g., less than the desired volume. Additionally, some existing devices use microfluidics, one-way valves, and pressure regulators.

Furthermore, as noted in the earlier introduction section of this disclosure, applying a significant amount of downward mechanical pressure (i.e., toward the subject) to a “push-to-actuate” type of actuator in a blood collection device may cause the blood collection device to slip or wobble and may potentially break a seal formed between the bottom of the device and the subject's skin. This may be especially relevant to blood collection devices with a height that is greater than a base dimension. Moreover, many people who might use a blood collection device have to collect blood relatively frequently and apply pressure frequently for the purpose of generating a vacuum. Applying significant pressure towards a subject's body may cause pain, bruising, or other damage to the subject and experiencing these problems, especially on a frequent basis, may discourage people from using such blood collection devices.

In contrast to existing devices, the blood collector module 102 disclosed herein uses an ergonomic design which does not require maintaining lower than atmospheric pressure prior to actuation and does not require the slide latch 136 to be depressed, pushed, or compressed towards the subject's body to provide the energy to generate a vacuum. Instead, the energy used to generate a negative pressure is stored in the precompressed volume expansion spring 210 and the slide latch 136 is merely actuated by a short, gently lengthwise sliding motion 238 toward the proximal end 203 of the transfer module 104 (e.g., sample tube 105) which is coupled to a collector transfer port 244 formed in a distal collector portion 214 of the collector base 252.

The blood collector module 102, as illustrated in FIGS. 2B and 2C, further includes a concave mid collector portion 224 formed in the collector enclosure 254 between the proximal collector portion 206 and the distal collector portion 214 of the collector enclosure 254 to facilitate secure holding of the blood collector module 102 between two digits of a hand 123.

In various implementations, the concave mid collector portion 224 further includes a pressure distribution channel 228 formed in the collector base 252 and configured to equalize reduced air pressure within the dynamic modular depressurization chamber 208, the blood extraction chamber 216, and a transfer module 104 coupled to the blood collector module 102.

With respect to the proximal collector portion 206, the distal collector portion 214, and the concave mid collector portion 224, these references are intended to convey the relative location of various parts of the collector base 252 and the collector enclosure 254 and should not be interpreted as describing strict boundary lines that define each portion.

Consistent with this understanding, it may be noted that the collector enclosure 254 encloses most of the collector base 252 (other than the sealing surface 232) even though in various example implementations they are formed or molded as separate parts which are configured to step together. Accordingly, references to structures or functions related to the proximal collector portion 206, the distal collector portion 214, and/or the concave mid collector portion 224 should be understood as referring to the respective relative positions of the described portions of the blood collector module 102.

By way of example, in the depicted example, the dynamic modular depressurization chamber 208, the blood extraction chamber 216, and the pressure distribution channel 228 are all at least partially hollow sections included and formed in the respective proximal, distal, and mid collector portions of the collector base 252. Referring to the general location of the dynamic modular depressurization chamber 208 as being in the proximal collector portion 206 and the general location of the blood extraction chamber 216 as being in the distal collector portion 214 helps clarify that unlike existing designs where the dynamic modular depressurization chamber 208 and the blood extraction chamber 216 overlap to some degree, in the blood collector module 102, the dynamic modular depressurization chamber 208 and the blood extraction chamber 216 are located in geometrically separate portions such that in order for the air pressure to be equalized in the dynamic modular depressurization chamber 208 and the blood extraction chamber 216, a pressure distribution channel 228 between the dynamic modular depressurization chamber 208 and the blood extraction chamber 216.

At the same time, the dynamic modular depressurization chamber 208, the blood extraction chamber 216, and the pressure distribution channel 228 are all at least partially enclosed by the collector enclosure 254 and may therefore be referred to as being included in or comprised within the respective proximal, distal, and mid collector portions of the collector enclosure 254. Similarly, separate components (e.g., the depressurization piston, O-rings, lancet carrier, and so forth) which are located at the respective proximal, distal, and mid collector portions of the collector enclosure 254 and of the collector base 252, may also be referred to as being included in or comprised within such respective proximal, distal, and mid collector portions of either the collector base 252 and of the collector enclosure 254, based on the fact that such separate components are at least partially enclosed within such portions of the collector enclosure 254 end of the collector base 252 and should not be understood as necessarily being formed as part of the collector enclosure 254 or of the collector base 252.

The concave mid collector portion 224 includes a pressure distribution channel 228 configured to equalize reduced air pressure within the dynamic modular depressurization chamber 208, the blood extraction chamber 216, and a transfer module 104 coupled to the blood collector module 102. During blood collection, when the blood collector module 102 is attached to the skin 122 of the subject 116, the pressure distribution channel 228 is higher than the collection opening 235 and the skin 122 of the subject 116 is sealed to the sealing surface 232 by the skin adhesive 233 so that in response to the slide latch 136 being actuated by the lengthwise sliding motion, the precompressed volume expansion spring 210 is released causing a depressurization piston 262 to move and a first negative pressure 326 to be distributed within the dynamic modular depressurization chamber, the blood extraction chamber 216, and the transfer module to facilitate rapid blood flow downwards to the transfer module 104, e.g., a sample tube 105.

It may be noted that in various implementations, the pressure distribution channel 228 distributes dynamically depressurized air to various regions of the blood collector module 102.

In some implementations, pressure distribution happens during an initial stage of blood collection and in a later stage the dynamic modular depressurization regulator provides different pressures in different sections of various modules such as a transfer module 104 (e.g., a sample tube 105) and/or a plasma separator module 106 (which may separably coupled as described with respect to FIGS. 3H and 3I or may integrally coupled with some modifications in pressure distribution as described with respect to FIGS. 4A and 4B).

Referring now to further improvements relating to illustrations intended to portray the accelerated blood flow of the apparatuses, modules, and methods disclosed herein.

FIG. 2J depicts a blood collector module 102 placed on and sealingly coupled to skin 122 at a collection site 120 of a body part such as an upper arm, of a subject 116. In various implementations, the plurality of linearly-arranged lancets strips 242 are configured to cause a plurality of rows of blood extraction slits 278 in the skin 122 to be generally aligned with a blood flow direction from the blood collector module 102 to the transfer module 104 to inhibit flow interference from edges of the blood extraction slits 278. In a simple example, depicting two rows of blood extraction slits 278, blood 126 is seen flowing from rows of blood extraction slits through the non-microfluidic blood flow channel 246 (shown in FIG. 2F) into the transfer module 104, which may be a sample tube 105.

Since there are various implementations at least one of which include an integrally coupled implementation of the blood collector module 102b described below with respect to FIGS. 4A and 4B, in very many respects, the elements and functions of the blood collector modules 102a, 102b and the plasma separator modules 106a, 106b are very similar with outwardly noticeable differences in the physical form factor and manner of coupling.

Accordingly, it should be noted that with respect to the integrally coupled blood collector module 102b around plasma separator module 106b, the blood extraction chamber 216 structure and everything inside it is adjusted toward the base by about the height of the plasma separator module 106b. In other words, because the height of the plasma separator module 106b is about 4 mm, and certain elements such as the lancet carrier need to extend down through the collection opening 435 in the plasma separator module 106b shown in FIG. 4B, certain elements need to be adjusted on the distal side of the blood collector module 102b to accommodate the additional height at the bottom portion of the blood collector module 102b where the plasma separator module 106b is integrally coupled.

For example, the blocking elements 271, and the actuation indicator 130 and various other components that will be recognized by the skilled in the art of device manufacturing should be adjusted by about the height of the plasma separator module 106b as described below with respect to FIGS. 4A-4B. Also, a sealing surface 432 in the blood collector module 102b is at the bottom of the integrally coupled plasma separator module 106b and corresponds to the sealing surface 232 of the blood collector module 102a.

FIG. 2J is an isometric bottom view of two different implementations of lancet strip assemblies 241a, 241b each with a plurality of linearly-arranged lancet strips 242 configured to allow blood to flow from a plurality of rows of blood extraction slits 278 through one or more channels between the linearly-arranged lancet strips 242. In some implementations, the linearly-arranged lancet strips 242 are configured in a parallel arrangement such as depicted for the lancet strip assembly 241a. In certain implementations, the lancet strip assembly 241b includes a converging arrangement of the linearly arranged strips 242 that helps channel the flow of blood to converge.

FIG. 2K is an enlarged perspective view of a lancet strip assembly 241 that includes a plurality of linearly-arranged lancet strips 242 arranged in a parallel arrangement. Such an arrangement facilitates blood flow within a channel between two lancet strips 242. It may be noted that although many of the examples illustrated herein depict and describe one or more lancets 213 as being configured as linearly-arranged lancet strips 242, certain implementations of the apparatuses, systems and methods, described or depicted herein as having linearly-arranged lancet strips 242 may also be implemented using a single lancet that has been designed to minimize pain e.g., by having one or more particularly sharp blade edges. In some embodiments, the apparatuses, systems, and methods described herein may be implemented using one or more lancets arranged in geometries other than linearly arranged lancet strips without departing from the benefits and improvements provided by the other components and combinations of components described and depicted throughout this disclosure.

FIG. 2L is an illustration of a top view, side view, and front view, of a lancet 213 such as may be used in some implementations of the plurality of linearly-arranged lancet strips 242. In some implementations, the lancets in the lancet strip assembly 241 are cut in flat patterns in the plane of a sheet of suitable metal such as biocompatible stainless steel using a laser machining system.

Laser machining or micromachining is a process that uses lasers to cut, drill, and shape materials on a very small scale. This method is beneficial for manufacturing microneedles or micro lancets, as it offers a high level of precision. When selecting a material, there are several factors to consider such as biocompatibility, machinability, strength and durability, and corrosion resistance. In various implementation, the inventors of the subject matter disclosed herein determined that stainless steel, titanium, and/or titanium alloys such as nitinol may be used to form the lancet strip assemblies 241. In one working example, lancet strips such as depicted in FIG. 2L can be used to fabricate lancets that are substantially painless upon penetrating skin and enable accelerated ergonomic collection of capillary blood in volumes 218 ranging as high as nearly 600 μL.

Although the typical collected blood volumes 218 of around 500 μL is relatively high for substantially painless blood collection devices, the inventors of the subject matter disclosed herein determined that by increasing the sharpness of the lancets, even greater volumes could be efficiently collected with accelerated times. However, conventional methods of sharpening using micromachining lasers or mechanical sharpening means is challenging due to additional machining time required to produce lancets with acceptable sharpness and uniformity between batches.

The inventors of the subject matter disclosed herein recognized a need to improve upon existing lancet sharpening technology.

By performing chemical etching with offset etching masks, lancets 213 such as depicted in FIG. 2M that are extremely sharp can be manufactured where at least a piercing portion of the lancets 213 includes single-bevel blade edges 219 instead of the rectangular blade edges 217 produced by laser machining or other micromachining techniques.

Beneficially, the graph of the results of the lancets 213 with single-bevel blade edges 219 shows an increase in the speed and the volume of blood collection consistent with the objective of providing accelerated ergonomic collection of capillary blood.

FIG. 2N includes a graph of a volumes 218 for different lancet blade edges that on the left-hand side shows rectangular blade edge volume results 220 of blood collection volumes 218 using one or more lancets 213 that are micromachine using lasers and have rectangular blade edges 217 with typical volumes of around 500 μL and as high as nearly 600 μL.

On the right-hand side, single-bevel blade results 222 of the graph of volumes 218 shows volumes collected using lancets 213 that have been chemically-etched to include single-bevel blade edges 219 are significantly higher with typical volumes in the range of 1000 μL of blood collected rapidly. Moreover, in some examples, the lancets 213 with single-bevel blade edges 219 reduce perceived pain.

FIG. 2O is an illustration of an onscreen communication 134 displayed on a portable electronic device 110 for a collection site preparation step for performing accelerated ergonomic collection of capillary blood, according to one or more examples of the disclosure. In some examples, prior to the act of coupling the blood collector module 102 to the region of skin 122 of a body part 117 (e.g., upper arm) of a subject 116, an onscreen communication 134 indicate that the step of including a blood flow agent 286 on the skin 122 of a body part 117 (e.g., upper arm) of a subject 116.

In various examples, prior to coupling the blood collector module 102 to the region of skin of a body part 117 (e.g., upper arm) of a subject 116, a blood flow agent 286. In certain examples, the blood flow facilitation agent includes a heat pack, a stream of warm water, a heated cloth, or similar heating agent. In various examples, the blood flow agent 286 may include an anticoagulant 288, a vasodilator 289, and/or a heating agent 290. In certain examples, the blood flow agent 286 includes lidocaine 292. Although lidocaine may have anesthetic effects as well, merely applying a topical anesthetic may be counterproductive. For example, some topical anesthetics such as those used for relief of tattoo pain and swell include lidocaine but also include epinephrine which is a vasoconstrictor which may reduce the volume and/or rate of blood collection.

FIG. 2P is an illustration of a non-microfluidic blood flow channel anticoagulant atomization protocol for accelerated ergonomic collection of capillary blood, according to one or more examples of the disclosure. For example, a thin coating of anticoagulant 288 may be applied through the angled collector transfer port 244 using atomization or any suitable coating technique to reach the non-microfluidic blood flow channel 246 of the blood collector module 102.

FIG. 2Q is a chart 295 of blood volume collected by blood flow agent type test results for different skin preparation protocols for accelerated ergonomic collection of capillary blood, according to one or more examples of the disclosure. A test of blood collection volumes was performed using early prototypes of laser cut lancets that did not include single bevel blade edges.

On a left portion of the chart 295, the volume results obtained are for a blood flow agent 286 that includes a heating agent 290 such as warm water or a warming object or substance. The results show that typical blood volumes collected ranged between about 115 microliters to 460 microliters which is higher than the volume that is collected when no blood flow facilitation agent is used.

On a right portion of the chart 295, the volume results are for a blood flow agent 286 that includes lidocaine 292. These results show that typical blood volumes collected ranged between about 190 microliters to 671 microliters which is higher than the volume that is collected when no blood flow agent 286 is used and is also even high than the volume collected using a heating agent 290, as the blood flow agent 286.

In various implementations, the effect of blood flow facilitation agents described and depicted with respect to FIGS. 2O, 2P, 2Q may be combined with the effect of lancet strips 242 with lancets 213 with single-bevel blade edges 219 for improved results in volume, collection rate, and reduced perception of pain to provide significant improvements over existing systems.

Now that a detailed description of many of the components of system 100 has been provided, it may be helpful to describe certain operational aspects of the system 100 and its components in more detail.

The accelerated ergonomic collection of capillary blood is significantly benefited by various mechanical features of the blood collector module 102. However, certain electronic, software, biochemical, and physical features may also enhance the accelerated ergonomic collection of capillary blood. For example, the process tracking module 108 may be configured to display various steps to be performed by the subject during the modular self-collecting of capillary blood to enhance accelerated blood flow and/or enhance economic usage of the blood collector module 102 and other components of the system 100.

For example, blood collection operational instructions (which may in certain examples be presented via onscreen communications 114 on the portable electronic device 110 such as depicted in FIG. 1) may be configured to provide beneficial instructions to the subject to prepare to collect the blood such as, running water over the arm from which blood is to be collected.

In some examples, a process tracking module 108 selectively displays certain instructions based on the type of transfer module 104 to be used, the type of test to be performed using the blood sample, and profile data for the particular subject. For example, an example operational instruction may describe how the blood collector module 102 is designed to be adhesively coupled to a region of skin 122 of a subject. In some implementations, the instructions may vary depending on the characteristics of the subject. For example, as mentioned above, for small children, a simple collection site may be found on a lower back region, or for animals, other body a suitable collection site may be found on vertical regions of skin which are shaved or otherwise hairless.

The blood collector module 102 is shipped with a release liner 237 (shown in FIG. 2A) which may be removed just prior to adhesively coupling the blood collector module 102 to the region of skin 122 of the subject.

In accordance with instructions provided by the laboratory or other home use blood collection kit provider, the subject follows instructions displayed by the process tracking module 108 (shown as a box with dashed lines in the portable electronic device 110 on FIG. 1 and described in more detail with respect to FIGS. 5, 6, and 7) to couple a transfer module 104 to the blood collector module 102 so that a collected blood level indicator such as a fill line on the blood collector module 102 or on the transfer module 104 is directly and readily visible within clear-sighted the subject as the subject faces towards the blood collection site of the region of skin. In some examples, the collected blood level indicator may be an onscreen communication that indicates sufficient blood has been collected based on elapsed timing of a time implemented by the process tracking module 108. As noted in the beginning of this application, modules such as the process tracking module 108 may be implemented or may include software components, hardware components or a combination of hardware and software components.

The angled collector transfer port 244 helps orient coupling of the transfer module 104 so that when coupled to the blood collector module 102 the distal end 207 of the transfer module 104 is lower than the proximal end 203 of the transfer module 104 which provides a gravity assist to aid the flow of blood 126 from the blood collector module 102 to the transfer module 104.

Moreover, with the blood collector module 102 adhesively coupled to a vertical region of skin 122 of the subject, the transfer module 104 or sample tube 105 is positioned at an outward angle relative to the sealing surface so that it may be positioned to be viewed over portions of the subject's body while remaining stably coupled. For example, in various implementations, the angled collector transfer port 244 is disposed distally of the blood collector module 102 and angled away from the subject at an acute angle of between 10 and 45 degrees relative to the sealing surface 232.

As depicted in FIG. 1, in implementations in which the plasma separator module 106b integrally coupled to the blood collector module 102b, the plasma separator module 106b is configured to output the plasma 128 to an angled collector transfer port the couples to a transfer module 104 such as a sample tube 105 or other transfer modules 104 with compatible coupling dimensions.

After the subject slides the slide latch 136 of the blood collector module 102 lengthwise towards the transfer module 104, he or she may tap the start button displayed in an onscreen communication 114, whereby, in certain examples, the process tracking module 108 determine a time that the blood collection was started for determining an approximate collection period. In various examples, the process tracking module 108 communicates with a data network 112 via the portable electronic device 110. Additional details about the structure and functions involved with the process tracking module 108 are described below with respect to FIGS. 5, 6, 7, and 8. After the blood is collected, the subject may remove the blood collector module 102 from the region of skin 122 and send the transfer module 104 to be appropriate receiving entity for further processing.

Referring again to FIGS. 2A-2C, in various examples, the blood collector module 102 includes a skin adhesive 233 on a sealing surface 232 of the collector base 252 of the blood collector module 102. In various examples, the blood collector module 102 is configured to stably couple to a region of skin 122 of a subject. Certain aspects of the blood collector module 102 enhance its stability when coupled to a region of skin. In one or more implementations, a stabilizing aspect is that the diameter of the collector base 252 is at least as large as the height of the blood collector module 102. When a subject slides the slide latch 136, the sealing surface 232 being relatively wide at a bottom surface of the collector base 252 inhibits the blood collector module 102 from tipping or rocking.

In various implementations, the skin adhesive 233 that forms a sealing surface 232 at the bottom of the collector base 252 is adhesively coupled to the bottom surface of the collector base 252 so that it covers an open bottom portion of a pressure distribution channel 228. The skin adhesive 233 (depicted as a dot pattern in FIG. 2A) for adhesively sealing a bottom surface of the collector base 252 to the skin 122 of the subject 116 during collection of the blood 126.

In certain implementations, when the blood collector module 102 is provided to a subject, the skin adhesive 233 is covered by release liner 237 (depicted with a tab pulled up in FIG. 2A) that the subject is instructed to remove immediately before sticking the blood collector module 102 onto the skin 122 of the subject 116.

Preceding portions of the detailed description have highlighted various ergonomic features of the system 100 and the blood collector module 102 itself. Additionally, various of the following portions of the detailed description provide further information about how certain features of the system 100 and the blood collector module 102 may be used to facilitate additional operations such as plasma separation for transfer in liquid form as well as accelerated collection of capillary blood. Such features include, for example, the number and arrangement of the lancet strips, the preparation of the subject and the modules, and the use of certain types of transfer modules to implement dynamic modular depressurization and related functions such as reagent mixing, plasma separation, and other useful features that accelerate efficient collection of capillary blood and/or plasma.

Referring to an aspect of acceleration of the blood extraction, FIG. 2E is a cross-sectional side view of a blood collector module 102 with a plurality of linearly-arranged lancet strips 242 for accelerated ergonomic collection of capillary blood, according to one or more examples of the disclosure.

In various examples, the blood collector module 102 includes a plurality of linearly-arranged lancet strips 242 which when triggered by a subject depressing the slide latch 136, is propelled downward to momentarily puncture and retract to produce micropunctures in the region of skin of the subject.

In certain examples, the plurality of linearly-arranged lancet strips 242 or some other arrangement of the lancets 213 is coupled to a lancet carrier 240 that is biased by a lancet carrier spring 248. Prior to actuation, the lancet carrier spring 248 is held in a compressed state by one or more lancet carrier latches 274 that clip onto a lancet carrier foot 275 of the lancet carrier 240. When the slide latch 136 is actuated by a lengthwise sliding motion toward the angled collector transfer port 244, the lancet carrier latches 274 are unlatched (e.g., pulled away from the lancet carrier foot 275 of the lancet carrier 240 by a collar spring 272 pushing an extraction collar 270 to which the lancet carrier latches 274 are coupled, away from the lancet carrier foot 275 of the lancet carrier 240 so that they no longer catch on the lancet carrier foot 275). The spring force of the lancet carrier spring 248 propels the lancet carrier 240 downward causing the plurality of linearly-arranged lancet strips 242 to momentarily puncture and retract from the region of skin 122 of the subject. The term “momentarily” as used herein refers to a very brief period of time such as less than 1000 milliseconds, less than 500 milliseconds, or less than 250 milliseconds.

By causing the plurality of lancet strips or other arrangement of lancets to rapidly puncture and retract from the region of skin 122 at the collection site 120, the likelihood of pain being perceived by the subject may be reduced. Furthermore, the faster that blood starts flowing from the micropunctures through the non-microfluidic blood flow channel 246 to the transfer module 104, the lower the likelihood is that the collected blood will start to clot.

In one or more implementations, there is a collection opening 235 in the sealing surface 232 of the blood collector module 102. The collection opening 235 forms a perimeter through which pass tips of a plurality of linearly-arranged lancet strips 242. More about the additional details about the plurality of linearly-arranged lancet strips 242 are described below with respect to FIGS. 2I, 2J, and 2K. The collection opening 235 is fluidically coupled to a non-microfluidic blood flow channel 246 that passes through the angled collector transfer port 244. In various examples, the non-microfluidic blood flow channel 246 is large enough that as blood 126 flows into the transfer module 104, the flow is self-venting meaning that no separate air return path is needed. In the depicted implementation, no separate air return path is needed.

One feature that helps facilitate accelerated and efficient blood flow of blood and plasma samples uncontaminated by hemolysis is the blood flow initiator strip 247 depicted and described with respect to FIG. 2G that also reduces the likelihood of closing off of air return paths within the non-microfluidic blood flow channel 246 due to adhesion forces of blood to portions of the blood flow channel wall 236. It may be noted however that certain implementations may include a non-microfluidic blood flow channel 246 with a separate air return path (not shown).

In general, there is an inverse relationship between blood flow channel resistance and the blood flow rate—the higher the blood flow resistance, the slower the blood flow rate. Slower blood flow rate may lead to increased coagulation and/or reduced blood volume collected. In some implementations, a maximum length of the non-microfluidic blood flow channel 246 is less than a minimum inner diameter of the transfer module 104. A shorter length of the non-microfluidic blood flow channel 246 minimizes the resistance. This represents a significant improvement over microfluidic designs where the length of the microfluidic channels is relatively long compared to an inner diameter of the collection container or a transfer module 104 of various types.

In various implementations, an inner minimum cross-sectional area of the non-microfluidic blood flow channel 246 in the blood collector module 102 is at least 30% as large as the minimum inner cross-sectional area of the transfer module 104 through which blood 126 collected using the blood collector module 102 is configured to flow.

In various examples, prior to the act of coupling the blood collector module to the region of skin of a body part 117 (e.g., upper arm) of a subject 116, a blood flow facilitation agent. In certain examples, the blood flow facilitation agent includes a heat pack, a stream of warm water, a heated cloth, or similar heating agent. In various examples, the blood flow facilitation agent includes an anticoagulant 288 and/or a vasodilator 289. In certain examples, the blood flow facilitation agent includes lidocaine. Although lidocaine may have anesthetic effects as well, merely applying a topical anesthetic may be counterproductive. For example, some topical anesthetics such as those used for relief of tattoo pain and swell include lidocaine but also include epinephrine which is a vasoconstrictor and would likely reduce the volume and/or rate of blood collection.

As explained in more detail below with respect to the detailed description of FIG. 2I, 2J, the blood collector module 102 punctures and retracts from the skin 122 of the subject, after which capillary blood begins to flow into the collection opening 235 and from there through the non-microfluidic blood flow channel 246 to the angled collector transfer port and transfer module 104 where it is collected.

One of the benefits of using a non-microfluidic blood flow channel for blood collection, such as the non-microfluidic blood flow channel 246, is that the submillimeter dimensions of microfluidic channels may be more susceptible to clogging due to blood clotting. Furthermore, the submillimeter dimensions of microfluidic channels may result in inadequate blood flow rate from the blood collector module 102 to the transfer module 104. In addition to prolonging the blood collection duration for the subject, slower blood flow rates may increase the likelihood of blood clots forming. Moreover, using microfluidic channels especially in the presence of a partial vacuum or pressurized air increase the likelihood of hemolysis or similar damage to blood 126. Accordingly, in various examples, the non-microfluidic blood flow channel 246 has minimum dimensions that are greater than submillimeter.

In some examples, the non-microfluidic blood flow channel 246 include a friction reducing (e.g., lubricious) material that facilitates the flow of blood 126 through the non-microfluidic blood flow channel 246. Various types of lubricious polymers may be used to form or coat the non-microfluidic blood flow channel 246. The non-microfluidic blood flow channel 246 may include a friction reducing material or coating. In some examples, the lubricious materials selected are also antithrombogenic to prevent formation of blood clots, which further facilitates smooth flow of the blood from the rows of blood extraction slits 278 in the skin 122 through the non-microfluidic blood flow channel 246 to the transfer module 104.

As depicted in the enlarged inset shown in FIG. 2J, as blood 126 from rows of blood extraction slits 278 are produced by the multiple linearly-arranged lancet strips 242 (as discussed below with respect to FIG. 2I, 2J) of the blood collector module 102 fill the collection opening 235, it flows through the non-microfluidic blood flow channel 246 towards the angled collector transfer port 244 which angles downward to a transfer module 104 thus displacing air which is in the transfer module 104. In turn, the displaced air flows above the blood 126 which equalizes the air pressure in the collection opening 235 and the transfer module 104 coupled to the blood collector module 102 so that the blood 126 can continue to flow down until the transfer module 104 is filled to a predetermined level.

FIG. 2D is a pre-actuation cross-sectional side view of a blood collector module 102 with a dynamic modular depressurization chamber 208 for accelerated ergonomic collection of capillary blood, according to one or more examples of the disclosure. In various examples, the dynamic modular depressurization chamber 208 holds a depressurization piston 262 that is biased by a precompressed volume expansion spring 210.

FIG. 2E depicts that in one or more examples, the slide latch 136 is mechanically coupled to the plurality of linearly-arranged lancet strips 242 and configured upon sliding lengthwise by a first predetermined distance toward the transfer module 104 to trigger a plurality of linearly-arranged lancet strips 242 to momentarily puncture and retract to produce rows of blood extraction slits 278 in the skin 122 of the subject 116.

Similarly, when the slide latch 136 is easily slid a second predetermined distance toward the transfer module 104, the precompressed volume expansion spring 210 is released and the volume of space within the dynamic modular depressurization chamber 208 begins to increase due to the depressurization piston 262 being pushed away from the sealing surface 232 thus reducing air pressure inside the expanded dynamic modular depressurization chamber 208 and at the collection opening 235 above the rows of blood extraction slits 278 relative to ambient pressure. This reduction of pressure above the rows of blood extraction slits 278 in the skin may beneficially assist in drawing blood out of the skin and into the angled collector transfer port 244.

In some implementations, a plurality of linearly-arranged lancet strips 242 are configured as two parallel rows such as depicted in FIGS. 2I, 2J. However, some implementations may be configured with more than two rows. In certain implementations, the linearly-arranged lancet strips 242, may be configured to have one or more V shapes such that the downstream or distal collector portions of the lancet strips 242 are closer together than the proximal collector portions of the lancet strips 242. This arrangement may in certain circumstances enhance the accelerated blood flow by guiding the blood flows from the individual lancet strips 242 to flow together.

Referring to the relative position of the slide latch 136 in the partially actuated state in which the lancet strips 242 are triggered to punch her the skin 122 of the subject 116 as depicted in FIG. 2E and the position of the slide latch 136 in the fully actuated state in which a first negative pressure is generated in the dynamic modular depressurization chamber 208 by the movement of the depressurization piston 262 and the O-ring 264 within the dynamic modular depressurization chamber 208 by the expansion of the precompressed volume expansion spring 210, these relative positions are mechanically selectable by using slide latches 136 that are differently configured in which the predetermined positions for generating the first negative pressure and/or triggering the lancet strips of the slide latch 136 are selected based on where the blocking elements of the slide latch 136 are formed relative to the spring-loaded elements that are held in place by the respective blocking elements of the slide latch 136.

In other words, by selecting a differently configured slide latch 136 the triggering of the dynamic modular depressurization piston and the lancet strips may be configured to occur with the lancet strips triggering first and the dynamic modular depressurization occurring second as depicted in FIGS. 2E and 2F. In other implementations, the triggering of the dynamic modular depressurization piston and the lancet strips may be configured to occur substantially at the same time. In further implementations, the triggering of the dynamic modular depressurization piston may be configured to occur prior to the triggering of the lancet strips by selecting a slide latch 136 configured to release the depressurization piston 262 pressing against the volume expansion spring 210 that is precompressed prior to releasing the extraction collar 270 to allow the lancet strips 242 to be triggered.

In some examples, the plurality of linearly-arranged lancet strips 242 comprises a plurality of lancet strips coupled to a lancet carrier 240 that is spring-loaded and that holds the rows of flat lancets latched in a retracted position prior to the lengthwise sliding of the slide latch 136; and propels the rows of flat lancets to momentarily puncture and retract from the region of skin 122 in response to the lengthwise sliding of the slide latch 136.

The inventors of the subject matter of the present disclosure have identified various factors that affect blood flow rate from wounds and thereby affect blood collection rate. Certain factors that increase blood flow rate may also increase the likelihood of a subject experiencing pain associated with the production of rows of blood extraction slits in the skin.

For example, a greater number of lancets in the linearly-arranged lancet strips may stimulate greater blood flow but may allow increase the likelihood of one or more of the lancets contacting one or more nerve endings thereby increasing the likelihood of the subject experiencing pain.

In certain implementations, such as for example, as depicted in FIGS. 2J,2K the linearly-arranged lancet strips 242 may include from about 3 to about 10 evenly distributed flat lancets of at least 1.5 mm in length to facilitate probabilistic penetration of a plurality of the lancets to a penetration depth that facilitates blood flow from sub-epidermis capillaries. As used herein the term “about” with reference to a whole number means plus or minus ten percent rounded to the nearest integer.

In certain implementations, a lancet 213 that is single or group of two or more lancets 213 configured as linearly arranged lancets strips 242 configured in non-linear arrangements may be selected or modified to have a high degree of sharpness which may significantly increase blood flow as described with respect to FIGS. 2M and 2N.

Getting a good blood flow rate for a blood is an initial objective but an efficacious system for accelerated ergonomic collection of capillary blood should include improvements to transfer modules and methods that facilitate rapid, safe, and efficient transfer of blood components for analysis and other uses.

FIG. 3A is an isometric top view of an apparatus 300 for accelerating efficient collection of plasma for transfer in liquid form using dynamic modular depressurization that includes a blood collector module 102 shown before actuation, a plasma separator module 106 and a transfer module 104, according to one or more examples of the disclosure. FIG. 3B is an isometric top view of an apparatus for accelerating efficient collection of plasma for transfer in liquid form using dynamic modular depressurization that includes a blood collector module 102 shown after actuation, a plasma separator module 106, and a transfer module 104, according to one or more examples of the disclosure.

In the example illustrated in FIGS. 3A and 3B, a plasma separator module 106 is coupled between the blood collector module 102 and a transfer module 104 which in this case is depicted as a sample tube 105 in which a significant volume of plasma for transfer in liquid form suitable for various tests may be efficiently collected by a subject 116 seamlessly initiating the process using a simple lengthwise sliding motion 238 of the slide latch 136 with the blood collector module 102 adhesively coupled to body part 117 (such as an upper arm or shoulder) of the subject 116.

As also depicted and described with respect to FIGS. 2C, 2F, a lengthwise sliding motion 238 of the slide latch 136 (e.g., in the distal/downward direction) actuates the blood collector module 102 and initiates a first stage of a dynamic modular depressurization process where the actuation is confirmed by the actuation indicator 130 changing indicating that the actuation has released the depressurization piston 262 to depressurize the dynamic modular depressurization chamber 208. The depressurization piston 262 rises causes a first negative pressure 326 to be distributed to the collection opening 235 in the blood collector module 102 to facilitate efficient flow of blood 126 from a collection site 120 on the body part 117 of the subject 116 as further depicted and described with respect to FIGS. 2C, 2F, and 2G.

FIG. 3C is an isometric bottom view of an apparatus 300 for accelerating efficient collection of plasma for transfer in liquid form using dynamic modular depressurization that includes a blood collector module 102 that has been decoupled from a collection site 120 on the body part 117 of a subject 116. The blood collector module 102 is coupled to a plasma separator module 106, which in turn is coupled to a transfer module 104 with collected plasma 128 for transfer in liquid form, according to one or more examples of the disclosure. Although the apparatus 300 is depicted with that blood collector module 102 separably coupled to the plasma separator module 106, in some implementations, the blood collector module 102 is integrally coupled to the plasma separator module 106 as depicted and FIGS. 4A and 4B. In various implementations, after decoupling the blood collector module from skin of the body part 117 of the subject 116, the dynamic modular depressurization enters a different stage of depressurization in response to a change in air pressure at the collection opening 235,435 which when decoupled from a collection site is at atmospheric pressure 329 i.e., P₀.

FIG. 3D is a cross-sectional view of an apparatus 300 for accelerating efficient collection of plasma for transfer in liquid form depicting a stage of dynamic modular depressurization in which upon actuation, air 127 flows upstream (which as used in this context generally refers to a direction towards the collection site 120 as opposed to downstream which, unless otherwise clear from context, generally refers to a direction away from the collection site 120 and towards a transfer module 104 e.g., where collected blood and/or plasma is stored to be transferred). The inventors of the subject matter disclosed herein have determined that in various implementations, a level of depressurization (e.g., negative air pressure at the collection site 120 and within one or more flow paths configured for moving blood 126 from the collection site 120 to a transfer module 104 such as a sample tube 105) for accelerated ergonomic blood collection (e.g., from a body part of a user such as a user's arm) ranges from about 32 kilo Pascals (kPa) to about 48 kPa less than current local atmospheric air pressure 329 i.e., P₀.

In the context of this disclosure, air pressures in a chamber or flow path within the range of 32 kPa to about 48 kPa are sometimes referred to as “negative pressures” meaning pressures that are less than current local atmospheric air pressure because a very broad estimate of “normal” current local atmospheric air pressures to within one standard deviation over all elevations and seasons, are generally in the range of about 65 kPa to about 104 kPa. Although this is a generalized range for normal atmospheric pressure and the actual atmospheric pressure can sometimes fall outside of this range due to certain unusual weather conditions the inventors of the all subject matter of this disclosure have determined that for purposes of providing a negative pressure that assists in collection of blood from a collection site, in various implementations, negative pressures in the range 32 kPa to 48 kPa below current local atmospheric air pressure are suitable.

In some implementations in which more intense negative pressures are used, a higher risk of hemolysis may result. In certain implementations in which less intense negative pressures are used, acceleration of blood collection may be diminished and clotting may occur.

However, in various existing systems, the inventors of the subject matter disclosed herein determined that if negative pressures in the range of 32 kPa to about 48 kPa below current local atmospheric air pressure were also used to depressurize to assist the flow of blood 126 through a plasma separation membrane, excessive force exerted on the red blood cells as they pass through the plasma separation membrane may cause red blood cells (RBCs) to burst. When hemolysis occurs and RBCs break open, releasing their contents into the surrounding plasma, the plasma specimen can be described as “hemolyzed.” The term used to describe this contamination of the plasma specimen due to the breakdown of RBCs is “hemolysis.” A hemolyzed sample can cause inaccuracies in laboratory test results, as the cellular contents released from the RBCs can interfere with the assays. The cellular contents released from hemolyzed RBCs cannot be as easily separated by the plasma separation membranes 317 as can RBCs because these cellular contents are smaller than RBCs and are thus not filtered out by microfiltration membranes. If a blood specimen shows evidence of significant hemolysis, a laboratory may reject the sample and request a new one.

To solve this problem, in various implementations, the apparatus 300 implements dynamic modular depressurization to provide a less intense negative pressure for assisting the plasma to flow through the plasma separator module 106. The apparatus 300 includes a plasma separator module 106 with a blood input port 302 that at least partially defines an upstream separator flow path 304 and couples to a non-microfluidic blood flow channel 246 of a blood collector module 102. The plasma separator module 106 also includes a plasma output port 306 that at least partially defines a downstream separator flow path 308 and is configured to transfer the plasma 128 in liquid form to a sample tube 105 with a predetermined outside diameter. The plasma separator module 106 further includes a separator body portion 309 that links the blood input port 302 and the plasma output port 306.

In certain implementations, the separator body portion 309 includes a blood transfer channel 313 (shown in FIG. 3I) that fluidically couples the upstream separator flow path 304 to an upstream entry surface 316 of one or more multilayer plasma separation units 310, the separator body portion 309 further comprising the downstream separator flow path 308 that fluidically couples a downstream exit surface 322 of the one or more multilayer plasma separation units 310 to the plasma output port 306 for performing dynamic modular depressurization of the upstream separator flow path 304 and the downstream separator flow path 308.

In FIG. 3D, small minus symbols are used to depict negative air pressures, such as in the region of the upstream separator flow path 304 and the downstream separator flow path 308. A curvy hollow arrow shows how air travels through passages and pairs of double lines that are dashed define outer regions of the upstream separator flow path 304 and the downstream separator flow path 308.

In contrast to existing systems, in various implementations, as depicted in FIGS. 3D, the plasma separator module 106 includes a dynamic modular depressurization regulator 324 that, in response to exposure of the upstream separator flow path 304 to a local atmospheric pressure 329 i.e., P₀ after it has been depressurized to a first negative pressure 326, reduces risk of hemolysis as plasma 128 is separated through the one or more multilayer plasma separation units 310 by regulating depressurization of the downstream separator flow path 308 to a second negative pressure 328 that does not go more than a predetermined limit below atmospheric pressure 329 i.e., P₀, where in some examples, a suitable predetermined limit is between about 7 kPa to 14 kPa below atmospheric pressure 329. This dynamic module depressurization occurs in stages.

FIG. 3D illustrates the beginning a of first stage in which since no air can enter the upstream separator flow path 304 because the blood collector module 102 is coupled to the collection site 120 such that an airtight seal is formed, when the 102 is actuated, as described with respect to FIGS. 2D-2F, the precompressed volume expansion spring 210 causes the dynamic modular depressurization chamber 208 to depressurize to a predetermined negative pressure which negative pressure is equalized via the pressure distribution channel 228 to the non-microfluidic blood flow channel 246 and then to the upstream separator flow path 304.

Negative pressure in the downstream separator flow path 308 is also equalized because air from the transfer module 104 e.g., sample tube 105 flows upstream through the separator body portion 309 (which includes plasma separation membranes 317 through which air can pass if dry rather than saturated with blood 126 where the membranes are included as layers in the one or more multilayer plasma separation units 310 that are described in more detail below with respect to FIGS. 3H and 3I). In FIG. 3D, groups of minus symbols indicate negative pressures that are lower than atmospheric pressure 329 as air 127 moves upstream as dynamic modular depressurization begins. At this stage, the negative pressure in both the upstream separator flow path 304 and the downstream separator flow path 308 (e.g., the sample tube 105) are substantially equalized.

In certain implementations, the plasma separator module 106 includes an access cover 332. In some implementations, the access cover 332 is used to insert or remove the dynamic modular depressurization regulator 324 during assembly or any other time that it needs to be accessed. In some implementations, various components of the blood collector module 102 and/or one or more downstream modules such as the plasma separator module 106 and or various transfer modules 104 may be configured to accommodate different ranges of negative pressures for the different modules based on typical differences in atmospheric pressure for particular application such as uses that are always at sea level or always at high altitude. In some implementations, the access cover 332 may be used to configure the dynamic modular depressurization regulator 324 to accommodate such differences in atmospheric pressure.

In various implementations, the blood collector module 102 and the plasma separator module 106 are designed to be efficient and ergonomic. Efficiency is further enhanced by utilizing the blood collector module 102 and the plasma separator module 106 as single-use products which also enhances their biological protection against contamination and/or incorrect use.

In FIG. 3D, it may be difficult to visualize a gasket opening 311 defined by the geometry of the upstream blood gasket 315 which allows air to flow upstream as part of the dynamic process so it may be helpful to refer to FIG. 3I in which the gasket opening 311 and the geometry of the upstream blood gasket 315 is more easily viewed in the exploded perspective view. Returning to FIG. 3D and FIG. 3E, pairs of long dash lines are used to depict outer portions of the upstream separator flow path 304 and pairs of long dash short dash lines are used to depict outer portions of the downstream separator flow path 308. Hand-drawn filled and curved arrows are used to symbolically represent the fact that air 127 flows from the transfer module 104 (e.g., sample tube 105) and through the plasma separator membranes 317 of the plasma separator module 106 to the upstream separator flow path 304 as the negative pressure is substantially equalized in all fluidically coupled compartments.

FIG. 3E is a cross-sectional view of an apparatus 300 for accelerating efficient collection of plasma form transfer in liquid form depicting a stage of dynamic modular depressurization in which, a first modular negative air pressure accelerates efficient flow of whole blood from a collection site to a plasma separation membrane, according to one or more examples of the disclosure. At this stage of the dynamic modular depressurization, negative pressure is still maintained as blood 126 flows from the collection site 120 to upstream separator flow path 304 of the plasma separator module 106 and from there to the separator body portion 309 where it enters the one or more multilayer plasma separation units 310 and makes contact with a portion of an upstream entry surface 316 or in other words makes contact with a portion of the plasma separation membrane 317 which begins to saturate with blood.

In a saturated state, air 127 does not flow upstream through the plasma separation membrane 317 of the one or more multilayer plasma separation units 310 and does not need to flow upstream because the stage the downstream separator flow path 308 is already substantially depressurized to the first negative pressure utilized to facilitate flow of blood from the collection site. Similarly, the dynamic modular depressurization regulator 324 is not actuated at this stage because the blood collector module 102 is still coupled to the collection site 120 so the upstream separator flow path 304 is also depressurized to the first negative pressure and is not exerting a differential pressure on the blood in the blood compartment layer 314 to cause the blood 126 to be pressurized against the plasma separation membrane 317.

When the amount of blood 126 reaches a predetermined level as indicated by a timer or by a visual or mechanical fill indicator on one or more of the blood collector module 102, the plasma separator module 106, a mobile application, the apparatus 300 including the blood collector module 102 and the plasma separator module 106 are decoupled from the collection site 120.

FIG. 3F is a cross-sectional view of an apparatus 300 for accelerating efficient collection of plasma 128 in liquid form depicting a stage of dynamic modular depressurization in which, a second modular negative air pressure accelerates efficient flow of plasma 128 but not RBCs through the plasma separation membrane 317 for collection in the transfer module 104 e.g., sample tube 105, according to one or more examples of the disclosure. In FIG. 3F, blood 126 has already entered the blood compartment layer 314 which is shared between a first multilayer plasma separation unit 310a and a second multilayer plasma separation unit 310b which operate in parallel in order to process plasma at twice the rate and volume of a single multilayer plasma separation unit 310.

FIG. 3G is an isometric view of an apparatus 300 that include an instance of a plasma separator module 106 for separation of plasma from capillary blood collected by a blood collector module 102 and transferred to a transfer module 104, e.g., a sample tube 105, according to one or more examples of the disclosure. By way of a brief overview, with the plasma already stored in the blood compartment layer 314, the upstream entry surface 316 of the plasma separation membrane 317 and fluidic contact with blood that is pressurized at atmospheric pressure 329 from the upstream separator flow path 304. The downstream exit layer of the plasma separation membrane 317 is pressurized at the second negative pressure which is limited to prevent hemolysis by the dynamic modular depressurization regulator 324 as depicted in FIG. 3G which causes a pressure differential across the plasma separation membrane of the first multilayer plasma separation units 310a and second multilayer plasma separation unit 310b to operate in parallel to separate the plasma 128 from RBCs in the blood 126. The plasma 128 then flows through the respective plasma output conduits 320b, 320a to the point that they converge and the plasma 128 is output in liquid form from the plasma output nozzle 323 which extends into the plasma output port 306 that couples to the transfer module 104 which in the depicted example is a sample tube 105.

A more detailed explanation of this stage of dynamic modular depressurization depicted in FIGS. 3F and 3G follows, when the blood collector module 102 is decoupled from the collection site, it may be placed in a stand 140 as the plasma separator module 106 enters the next stage of dynamic modular depressurization. The collection opening 235 is now exposed to atmospheric pressure.

The plasma separator module 106 includes a dynamic modular depressurization regulator 324 that, in response to exposure of the upstream separator flow path 304 to atmospheric pressure after it has been depressurized to a first negative pressure 326, reduces risk of hemolysis as plasma 128 is separated through the one or more multilayer plasma separation units 310 by regulating depressurization of the downstream separator flow path 308 to a second negative pressure 328 that does not go more than a predetermined limit 330 below atmospheric pressure.

One example implementation of a dynamic modular depressurization regulator 324 is an umbrella valve that opens as depicted in FIG. 3F when the pressure on the upstream side exceeds the pressure on the downstream side by more than a predetermined limit.

For example, assume that the atmospheric pressure at a particular location and time is about 100 kPa. the inventors of the subject matter herein determined that in various implementations, to diminish hemolysis during separation of plasma using the plasma separation membranes with application of negative air pressure, a predetermined limit of between about 7 kPa to 14 kPa below atmospheric pressure is suitable. Accordingly, the dynamic modular depressurization regulator 324 in such an implementation may be implemented as an umbrella valve that opens at a differential pressure of 14 kPa which would provide a second negative pressure 328 of 86 kPa in the downstream separator flow path 308 and a differential negative pressure with air pressure on the downstream side of the plasma separation membranes 317 in the one or more multilayer plasma separation units 310 being about 14 kPa lower than the air pressure on the upstream side of the plasma separation membranes 317 thus accelerating separation of the plasma and at the same time minimizing the risk of hemolysis. In certain examples, by using dynamic modular depressurization with the plasma separator module 106b, a percentage of plasma 128 collected within 5 minutes of for a sample of blood 126 of between 400 l and 500 l exceeds 70 percent of available plasma with less than 2% hemolysis.

As depicted in FIG. 3G, as long as blood 126 is contained in the blood compartment layer 314 and saturating the plasma separation membranes 317 of the one or more multilayer plasma separation units 310a, 310b the air pressure on the upstream side of the plasma separation membranes 317 will continue to cause the plasma 128 to drop/flow from the plasma output nozzle 323 into the transfer module 104 e.g., the sample tube 105.

A significant number of modern laboratory tests may be performed on plasma samples of 600 μL of plasma. By operating multiple multilayer plasma separation units 310a, 310b in parallel with dynamic modular depressurization, the speed and volume of plasma sample collection may be significantly improved over what could be achieved using existing plasma collection approaches whether at home, point of care, or lab-based because all operations are performed by the apparatus 300 (blood collector module 102 and plasma separator module 106) with no additional instruments or processing. and the entire volume needed may be saved to a sample tube in liquid form in real-time from blood collected essentially painlessly at home by a new user using an easy sliding motion of one finger in about three to five minutes. The user can then screw a cap on the sample tube, confirm needed information using a mobile application, and return the plasma sample in the sample tube via specially designed prepaid return packaging that ensures the safety and integrity of the sample as it is transferred to a laboratory for analysis.

FIG. 3H is a cross-sectional view of an apparatus 300 and a plasma separator module 106 with an enlarged inset view depicting selected components, according to one or more examples of the disclosure. It may be noted that when referring to the first and second multilayer plasma separation units 310, depicted in FIGS. 3H, 3I, layers or elements may be named using the singular form and numbered respective using ‘a’ and ‘b’ suffixes in order to emphasize that the term “one or more plasma separation units” in some implementations refers to a single plasma separation unit and in other implementations refers to two, three, four, five, or some other number of multilayer plasma separation units 310. Similarly, a description of an element with an ‘a’ suffix should be understood to also apply to an element with a ‘b’ suffix unless otherwise clear from context.

The enlarged inset in the upper right portion of FIG. 3H shows how, in various implementations, the apparatus 300 includes a plasma separator module 106 with a first multilayer plasma separation unit 310a and a second multilayer plasma separation unit 310b where the upstream portions of the first and second multilayer plasma separation units are together and share a blood compartment layer 314 and the downstream portions of the first and second multilayer plasma separation units 310a, 310b are apart and the plasma output conduit 320b of the second multilayer plasma separation unit 310b is extended to connect with the plasma output conduit 320a of the first multilayer plasma separation unit 310a.

Shown in FIG. 3H is a second enlarged inset view on the lower right-hand side in which a cross-sectional view of a drainage layer 319a of the second multilayer plasma separation unit 310a is depicted. As plasma is separated on the downstream exit surface 322 of the plasma separation membrane 317, it passes through an opening in the downstream plasma gasket 318a and from there is collected into the drainage layer 319a.

The dendritic cross-channel 327a in the drainage layer 319a is one of many channels that perpendicularly traverses the main drainage channel to provide efficient drainage of the plasma from the membrane into a plasma output conduit 320a which converges with plasma output conduits from other of the one or more multilayer plasma separation units 310 to output the plasma 128 to the plasma output nozzle 323 that is part of the plasma output port 306 that couples to the transfer module 104, which in this example is a sample tube 105.

Also visible in the inset in the lower left of FIG. 3H is a geometry of the upstream blood gasket 315 that blocks a flow of blood in a downstream direction from contacting edges of the plasma separation membrane 317. In various implementations, the one or more plasma separation membranes 317a, 317b or the one or more multilayer plasma separation units 310a, 310b, and so forth includes an asymmetric microfiltration membrane that includes one or more of polyethylene terephthalate, polysulfone, polyethersulfone, polyimide, and combinations thereof. One source of suitable asymmetric microfiltration membranes is Cytiva, 100 Results Way, Marlborough, MA.

The multilayer plasma separation units 310 include upstream membrane blood gaskets with a geometry that blocks the blood 126 from contacting edges of the plasma separation membrane 317 to minimize potential issues such as edge leakage, uneven flow distribution, compromised filter integrity, reduced lifespan, flow dead zones, and similar issues that could occur if certain existing microfiltration gaskets were used.

FIG. 3I is an exploded view of an apparatus 300 and a plasma separator module 106 depicting selected layers and structures and flows, according to one or more examples of the disclosure.

As previously described with regard to FIGS. 3D-3G in various implementations, the plasma separator module 106 includes a blood input port 302 that at least partially defines an upstream separator flow path 304 and couples to a non-microfluidic blood flow channel 246 of a blood collector module 102 and a plasma output port 306 that at least partially defines a downstream separator flow path 308 and is configured to transfer the plasma 128 to a sample tube 105 with a predetermined outside diameter. The exploded view of FIG. 3I illustrates in greater detail, the structures and functions of a separator body portion 309 that links the blood input port 302 and the plasma output port 306.

The separator body portion 309 also includes the downstream separator flow path 308 that fluidically couples a downstream exit surface 322 of the one or more multilayer plasma separation units 310 to the plasma output port 306 for performing dynamic modular depressurization of the upstream separator flow path 304 and the downstream separator flow path 308.

The downstream separator flow path 308 may be dynamically regulated to a different negative pressure to accelerate plasma separation through the one or more multilayer plasma separation units 310. At a high-level, the separator body portion 309 channels the blood 126 from the upstream separator flow path 304 to an upstream entry surface 316 of one or more multilayer plasma separation units 310 in which an upstream blood gasket 315, an upstream entry surface 316, a plasma separation membrane 317, a downstream plasma gasket 318, a drainage layer a 319, a plasma output conduit 320, and a downstream exit surface 322 are depicted and shown in an exploded view for each multilayer plasma separation unit 310.

In the depicted example, two multilayer plasma separation units 310a, 310b are depicted as operating in parallel in order to process plasma at twice the rate and volume of a single multilayer plasma separation unit 310. However, as previously explained, two, three, four, five, or some other number of multilayer plasma separation units 310 may be implemented using similar structures and functions.

The dynamic modular depressurization is facilitated by a dynamic modular depressurization regulator 324 that, in response to exposure of the upstream separator flow path 304 to atmospheric pressure after it has been depressurized to a first negative pressure 326, reduces risk of hemolysis as plasma 128 is separated through the one or more multilayer plasma separation units 310 by regulating depressurization of the downstream separator flow path 308 to a second negative pressure 328 that does not go more than a predetermined limit 330 below atmospheric pressure.

As further illustrated in FIG. 3I, the upstream portions of the first and second multilayer plasma separation units 310a, 310b are arranged proximate to each other and share a blood compartment layer 314 and the downstream portions of the first and second multilayer plasma separation units 310a, 310b are arranged from each other and the plasma output conduit of the second multilayer plasma separation unit 310b is extended to fluidically coupled with the plasma output conduit 320b of the first multilayer plasma separation unit 310a.

In various implementations, the ergonomically efficient modular design of the one or more multilayer plasma separation units 310 can be used in different form factors, such as for example as described with respect to FIGS. 4A and 4B. Other example implementations may include one multilayer plasma separation unit 310, three, four, five, or some other number of multilayer plasma separation units 310 operating parallel with a system of parallel output conduits delivering plasma separated in parallel to one or more transfer modules 104 such as for example, one or more sample tubes 105.

The exploded view of the multilayer first and second multilayer plasma separation units 310a, 310b provides additional details about the flow paths for the dynamic modular depressurization of air 127 and the separation of plasma 128 from 126 and the layers that facilitate such operations.

As also described with respect to the enlarged inset in the upper right-hand of FIG. 3H, the upstream portions of the first and second multilayer plasma separation units 310a, 310b are arranged proximate to each other and share a blood compartment layer 314. The blood compartment layer 314 holds blood 126 coming from the upstream separator flow path 304, which in this implementation is through a blood transfer channel 313 shaped like an inverted U-shape so that surface tension at the narrow ends of the inverted U-shape can keep the blood flowing through the bottom portion of the blood transfer channel 313 allowing air 127 to return through the upper portion of the inverted U-shape.

In some implementations, the blood compartment layer 314 is a molded component with a thickness of about 2 mm, a generally elliptical rim of about 2 to 3 mm and an arm extending longitudinally inward from one end across the opening through which a plasma output conduit 320b is defined by a series of precisely aligned holes in the layers that are stacked adjacent to each other.

In the depicted implementation, the next layer of the multilayer plasma separation units 310a, 310b is an upstream blood gasket 315a, 315b layered adjacent to the blood compartment layer 314. As described with respect to the enlarged inset view at the lower right-hand of FIG. 3H, a geometry of the upstream blood gasket 315a, 315b blocks a flow of blood 126 in a downstream direction from contacting edges of the plasma separation membrane 317 to minimize potential issues such as edge leakage, uneven flow distribution, compromised filter integrity, reduced lifespan, flow dead zones, and similar issues that could occur if certain existing microfiltration gaskets were used.

The next layer depicted in FIG. 3I is a plasma separation membrane 317 layered adjacent to the upstream blood gasket 315. When the plasma separation membrane 317a, 317b is saturated with blood 126, the upstream entry surface 316a, 316b defines a boundary of the upstream separator flow path 304 in the saturated state, air 127 does not flow through the plasma separation membrane 317a, 317b and with the blood input port 302 of the plasma separator module 106 exposed to atmospheric pressure e.g., when the blood collector module 102 is decoupled from the collection site 120 the upstream entry surface 316a, 316b is also at atmospheric pressure.

Opposite to the upstream entry surface 316a, 316b of the plasma separation membranes 317a, 317b is the downstream exit surface 322a, 322b from which separated plasma exits the membrane. In some context herein, the upstream entry surface 316a, 316b and the downstream exit surface 322a, 322b are also referred to as pertaining to the respective multilayer plasma separation units 310a, 310b since they respectively define certain boundaries for the blood 126, the plasma 128, the upstream separator flow path 304, and the downstream separator flow path 308.

In the depicted example of FIG. 3I, the next layer is a downstream plasma gasket 318a, 318b layered adjacent to the plasma separation membrane 317a, 317b. The geometry of the interior rim of the downstream plasma gasket 318a, 318b does not necessarily need to block the plasma 128 from contacting the edges of the plasma separation membrane 317a, 317b because negative pressure in the downstream separator flow path 308 pulls the plasma 128 away from the plasma separation membrane 317a, 317b to a drainage layer 319 layered adjacent to the downstream plasma gasket 318 where the drainage layer 319 is configured to channel the plasma 128 to a plasma output conduit 320a, 320b.

FIG. 4A is a cross-sectional view of an apparatus 400 with plasma separator module 106b integrally coupled with a blood collector module 102b and enlarged inset views depicting selected layers and structures, according to one or more examples of the disclosure.

As previously described, the apparatuses and modules thereof may be implemented in various forms by a person skilled in the art taking into account the guidance provided in the specification concerning the problems to be solved and the general approaches disclosed by the inventors of the present disclosure.

When referring to the first and second multilayer plasma separation units 410 depicted in FIGS. 4A and 4B, layers or elements may be named using the singular form and numbered respective using ‘a’ and ‘b’ suffixes in order to emphasize that the term “one or more plasma separation units” in some implementations refers to a single plasma separation unit and in other implementations refers to two, three, four, five, or some other number of multilayer plasma separation units 410. Similarly, a description of an element with an ‘a’ suffix should be understood to also apply to an element with a ‘b’ suffix unless otherwise clear from context.

In various advantageous example implementations as depicted in FIG. 4A, the apparatus 400 with plasma separator module 106b is integrally coupled with a blood collector module 102b. In many respects, the apparatus 400 is quite similar to the apparatus 300 with the plasma separator module 106a separable coupled with the blood collector module 102b with the outwardly noticeable differences being primarily, but not exclusively, in the physical form factor and manner of coupling.

Along with these outwardly noticeable differences there are a few other differences. For example, in the enlarged inset view on the lower right-hand side of FIG. 4A, the plasma separator module 106b includes a first multilayer plasma separation unit 410a and second multilayer plasma separation unit 410b that in many aspects are substantially similar to the first multilayer plasma separation unit 310a and second multilayer plasma separation unit 310b described with respect to FIGS. 3H and 3I. In response to the collection opening being exposed to an atmospheric pressure P₀ 429 after the blood collection is completed and the plasma separator module 106b is decoupled from the collection site 120, the dynamic depressurization regulator regulated a downstream regulator flow path 408 to a second negative pressure 428 that is no more than a predetermined limit below atmospheric pressure P₀ 429.

To assist in retaining collected blood within the multilayer plasma separation units 410a, 410b when the apparatus 400 is decoupled from the collection site 120 on a body part of the user, the blood compartment layer 414 which is shared by the first and second multilayer plasma separation units 410a and 410b includes an absorbent material 412 not utilized in the multilayer plasma separation units 310a, 310b for the separably coupled plasma separator module 106a.

A base plate 452 for the plasma separator module 106b corresponds substantially to the collector base 252 a modification that one or more plasma output conduits 420b, 420a may be formed in or connected to the collector base 252 to allow collected plasma 128 to flow downstream to the plasma output port 406 which corresponds to the collector transfer port 244 of the blood collector module 102b.

Starting with the blood collector module 102b on the left-hand side of FIG. 4A, the plasma output port 406 has a similar form factor to the angled collector transfer port 244 previously described with respect to the blood collector module 102a (e.g., without the integrally coupled plasma separator module 106b) except that instead of outputting the blood 126 to the transfer module 104 e.g., sample tube 105, the plasma output nozzle 423 is disposed to output the plasma 128 in liquid form via the plasma out port 406 to a transfer module 104 such as a sample tube 105.

FIG. 4B is an exploded isometric view of an apparatus 400 with a plasma separator module 106b integrally coupled with a blood collector module 102b for accelerating efficient collection of plasma 128 for transfer in liquid form depicting upstream airflow and downstream liquid flow through selected layers and structures, according to one or more examples of the disclosure.

Depicted in the center of FIG. 4B are the first and second multilayer plasma separation units 410a, 410b which correspond to similarly numbered layers of the first and second multilayer plasma separation units 310a, 310b and perform substantially the same functions as described above with respect to FIGS. 3H and 3I. Due to differences in form factor of the integrally coupled plasma separator module 106b, the flow paths of the blood 126, air 127 and plasma 128 are slightly modified relative to those depicted in FIG. 3I.

The downstream separator flow path 408 may be dynamically regulated to a different negative pressure to accelerate plasma separation through the one or more multilayer plasma separation units 410. At a high-level, the separator body portion 409 channels the blood 126 from the upstream separator flow path 404 to an upstream entry surface 416 of one or more multilayer plasma separation units 410 in which an upstream blood gasket 415, an upstream entry surface 416, a plasma separation membrane 417, a downstream plasma gasket 418, a drainage layer a 419, a plasma output conduit 420, and a downstream exit surface 422 are depicted and shown in an exploded view for each plasma separation unit 410.

In the depicted example, two multilayer plasma separation units 410a, 410b are depicted as operating in parallel in order to process plasma at twice the rate and volume of a single multilayer plasma separation unit 410. However, as previously explained, two, three, four, five, or some other number of multilayer plasma separation units 410 may be implemented using similar structures and functions.

As depicted in FIG. 4B, in various implementations, the first and second multilayer plasma separation units 410a, 410b include a blood compartment layer 414 at the center which is shared and which includes an absorbent material 412 that assists in storing the blood 126 within the blood compartment layer 414 which then flows through the upstream blood gasket 415a, 415b which blocks the blood from contacting edges of the plasma separation membrane as it flows to the upstream entry surface 416a, 416b of the plasma separation membrane 417a, 417b as negative pressure on the downstream exit surface 422a, 422b of the plasma separation membrane 417a, 417b assists in drawing the plasma 128 through the plasma separation membrane 417a, 417b. The plasma flows through the downstream plasma gasket 418a, 418b to be collected in the drainage layer 419a, 419b which includes a longitudinal channel and multiple dendritic cross-channels similar to drainage layer 319a, 319b. In some implementations the drainage layer includes a circular channel depicted as an outer ring through which plasma 128 collected from the plasma separation membrane 417a, 417b (shown as a dotted line) flows around the opening corresponding to the collection opening 435 above the collection site 120 from which blood is collected.

Next is the plasma separator base plate 452 which because the plasma separator module 106b is integrally coupled at the bottom of the 102b takes the place of the collector base 252 and includes a sealing surface 432 and a skin adhesive 433 as well as a collection opening 435 that respectively correspond from an operational perspective to the sealing surface 232 skin adhesive 233 and opening 235 described previously. A collector base 252 and a plasma separator top plate 427 provide flows paths or conduits for the air, blood, and, plasma to flow to and from the integrally coupled plasma separator module 106b illustrated in FIG. 4B.

In some aspects, a blood input port 402 for the plasma separator module 106b with the collection opening 435 performs similar functions to the collection opening 235 and the non-microfluidic blood flow channel 246 of the blood collector module 102a due to the integrally coupled nature of the plasma separator module 106b with the blood collector module 102b.

FIG. 5 is a schematic block diagram of an apparatus 500 that includes a portable electronic device 110 for implementing accelerated ergonomic collection of capillary blood, according to one or more examples of the disclosure.

The portable electronic device 110 may be a cell phone, a smart phone, tablet, a laptop computer, or similar device. The portable electronic device 110 in various examples includes a processor 504, a memory 506 that contains code and/or data related to accelerated ergonomic collection of capillary blood. For example, in certain examples, the process tracking module 108 is implemented as code executable by the processor 504. In various examples, the portable electronic device 110 includes an input interface 512 and an output interface 514 in some examples, a touchscreen display provides some input interface functions and some output interface functions.

In some examples, the portable electronic device 110 includes a communication interface 119 that may include one or more transceivers for such as mobile telecommunications transceivers, Bluetooth transceivers, and so forth.

In various examples, the portable electronic device 110 includes an NFC scanner 113 which is used by the process tracking module 108 to perform NFC scans of NFC tags 135 attached to the blood collector module 102 and/or to the transfer module 104. In some examples, a subject may initiate scanning by touching a display button labeled “scan NFC” and/or by tapping the portable electronic device 110 lightly against the NFC tag 135.

In some examples, the portable electronic device 110 includes a biometric scanner 518 that is accessible by the process tracking module 108 one or more optical cameras, and software for analyzing fingerprints, iris prints, facial recognition, and/or other biometric measurements that may be useful for confirming the identity of the subject. Additional detail regarding the structure and function of the blood collector module 102 is provided throughout this disclosure and in particular with respect to FIGS. 2A, 2B, 2C, 2D, 2E, 2F, 2G, 2H, 2I, 2J, 2K, It may be noted that although various advantages are provided by the inclusion in the system 100 of the process tracking module 108, in some examples, the blood collector module 102 and the transfer modules 104 are usable for collecting blood in a manual mode without the inclusion of the process tracking module 108.

FIG. 6 is an illustration of a subset 600 of onscreen communications 114 related to confirming proper use of the system for accelerated ergonomic collection of capillary blood, according to one or more examples of the disclosure.

Depicted in FIG. 6 is an onscreen communication 137, in which a user is instructed to enter the name of the subject 116 from whom blood is being collected e.g., using the system 100. In some example implementation, the user is further instructed to scan each module being used such as the blood collector module 102, the transfer module 104, and/or the plasma separator module 106. Scanning the modules may be done using an NFC scanner such as the NFC scanner 113 depicted in FIG. 5.

A further onscreen communication 138 include information selected from a manufactured date of the one or more selected modules, a expiry date of the one or more selected modules, a product identification number of the one or more selected modules, a device type of the one or more selected modules, blood collection timing information for the one or more selected modules, blood preservation and storage parameters for the one or more selected modules, shipment parameters for the one or more selected module, and combinations thereof. Communicating blood collection parameters 605, such as for example, those listed in onscreen communication 137, 138 of FIG. 6 or other parameters related to blood collection, enables the subject to determine whether the information matches expected information conveyed by others such as healthcare providers, requesting laboratory, insurer, and so forth.

FIG. 7 is an illustration of a group 700 of onscreen communications associated with preparation, actuation, confirmation, and sample transportation steps for accelerated ergonomic collection of capillary blood, according to one or more examples of the disclosure. In various examples, operational instructions such as those depicted in group 700 may be presented as onscreen communications 114 (such as 702, 704, 706, 708, 710, and 712 (or in certain implementations may be presented as printed instruction panels) for tracking accelerated ergonomic collection of capillary blood, according to one or more examples of the disclosure.

The onscreen communications may relate to preparation of the subject, preparation of the blood collector module or other apparatuses, operation and/or actuation of the blood collector module or other apparatuses, confirmation the subject, the collected blood and/or the apparatuses used to collect the blood or to separate plasma or perform other accelerated ergonomic collection of capillary blood. Certain instructions may provide information about sample transportation steps for accelerated ergonomic collection of capillary blood, according to one or more examples of the disclosure.

In various examples, a module preparation onscreen communication 702 describes an example of a blood collector module preparation step for performing accelerated ergonomic collection of capillary blood. Although the depicted example in the module preparation onscreen communication 702 describes removing a release liner from the blood collector module, other module preparation steps may be communicated such as information helpful to confirm that the right type of transfer module or sample tube is prepared and available to couple to the collector transfer port of the blood collector module. In some examples, the blood collector module preparation steps may include information about and packaging, handling of the blood collector module.

The instructions may also include information about the selection of a suitable collection site based on the type of subject from whom blood is being collected. For example, if the subject is an animal, the onscreen communication 702 may provide information about various locations on different body parts of the animal that may provide optimal results for blood collection. This could include information such as how to prepare the blood collector module to stick to the skin of the subject animal at a selected collection site.

A second depicted onscreen communication 704 provides information related to placing the blood collector module at the collection site.

A third depicted onscreen communication 706 provides information related to actuating the blood collector module placed at the collection site.

A fourth depicted onscreen communication 708 provides information related to biometric scanning to confirm information about the person on which the blood collector module is used.

A fifth depicted onscreen communication 710 provides information related to spill-free decoupling of the transfer module from the blood collector module.

A sixth depicted onscreen communication 712 provides information related to transportation of the transfer module.

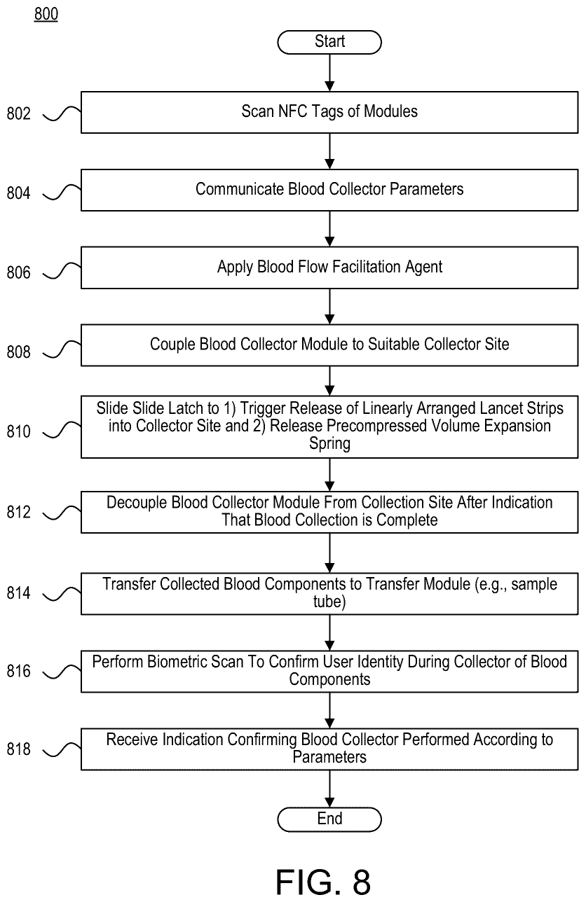

FIG. 8 is schematic flow chart diagram of a method 800 of accelerated ergonomic collection of capillary blood and plasma, according to one or more examples of the disclosure. In various implementations, the method 800 begins and includes scanning 802 an NFC tag coupled to one or more modules for blood collection. For example, as depicted in a module preparation onscreen communication 702 shown in FIG. 7, the subject may enter her name and may be instructed to scan NFC tags of a blood collector module and a transfer module that will be used to collect and transfer the blood (e.g., for processing at a laboratory).

The method 800 continues and in certain examples, includes communicating 804 one or more blood collection parameters to a subject undergoing blood collection. For example, as depicted in onscreen communication 137, 138 in FIG. 6, in certain implementations, the blood collection parameters include information selected from a manufactured date of the one or more selected modules, a expiry date of the one or more selected modules, a product identification number of the one or more selected modules, a device type of the one or more selected modules, blood collection timing information for the one or more selected modules, blood preservation and storage parameters for the one or more selected modules, shipment parameters for the one or more selected module, and combinations thereof. Communicating blood collection parameters 605, such as for example, those listed in onscreen communication 137, 138 of FIG. 6 or other parameters related to blood collection, enables the subject to determine whether the information matches expected information conveyed by others such as healthcare providers, requesting laboratory, insurer, and so forth.

In certain examples, the method 800 continues and includes applying 806 a blood flow facilitation agent to the region of skin of the subject to which the blood collector module will be coupled for blood collection.

One or more examples of a blood flow facilitation agent include a heat pack, a stream of warm water, a heated cloth, or similar heating agent. For example, certain types of reusable heat packs called snap heat pads, click heat pads, thermopacks, or similar, contain phase change materials such as sodium acetate that is in a supercooled liquid state at room temperature so that when a metallic triggering device such as a metal disc within the pad is flexed, the supercooled liquid begins to freeze and releases heat thus warming up to a temperature of about 54° C. Such heat packs are non-toxic, reusable, and widely available and may be packaged so as to act as a safe and consistent blood flow facilitation agent.

In various examples, the method 800 include applying 806 a blood flow facilitation agent comprising an anticoagulant 288 and/or a vasodilator 289 to the skin 122 at or around the collection site 120 prior to the act of coupling 808 the blood collector module to the region of skin of a body part 117 (e.g., upper arm) of a subject 116. In certain examples, the blood flow facilitation agent includes lidocaine. Although lidocaine may have anesthetic effects as well, merely applying a topical anesthetic may be counterproductive. For example, some topical anesthetics such as those used for relief of tattoo pain and swell include lidocaine but also include epinephrine which is a vasoconstrictor and would likely reduce the volume and/or rate of blood collection.

In one or more examples, the method 800 continues and include coupling 808 a blood collector module to a region of skin of a subject with the body part 117 (e.g., upper arm) of a subject 116 resting in a substantially horizontal position. In some example implementations, the blood collector module 102 is sealed during blood collection to the region of skin of the subject using an adhesive. In certain examples, the blood collector module includes a strap, band, cuff, sleeve, cincture, or similar flexible fastening mechanisms that facilitates secure coupling of the blood collector module to the region of skin of the subject.

In some examples, the flexible fastening mechanism enables greater blood flow rate and/or volume to be achieved during collection, e.g., by allowing the subject to move the arm in a pattern that facilitates blood flow, such as for example, a circular motion, a windmill motion, arm circumduction, shoulder circumduction, a swinging motion, or similar motion, immediately before or during (e.g., after the step of coupling 808 the blood collector module to a suitable collection site as described below) collection of blood using the blood collector module.

Positioning the blood collector module 102 horizontally or at an acute angle relative to a surface on which the arm rests facilitates easier actuation of the blood collector module by providing a stable support for the arm of the subject which in turn provides stable support for the blood collector module 102. Furthermore, such positioning enables the blood collector module 102 to be directly and readily visible within clear-sighted the subject as the subject faces towards the collection site 120 in the skin 122 of the subject 116. Likewise, as illustrated in FIG. 2A, a transfer module 104 may be connected to the angled collector transfer port 244 so that the blood flow is assisted by gravity or by centrifugal force.

In various examples, the method 800 continues and includes sliding 810 the slide latch 136 of the blood collector module 102 with a lengthwise motion towards a transfer module 104 and triggering a momentary puncture and retraction to produce multiple rows of blood extraction slits in the region of skin 122 of the subject 116.

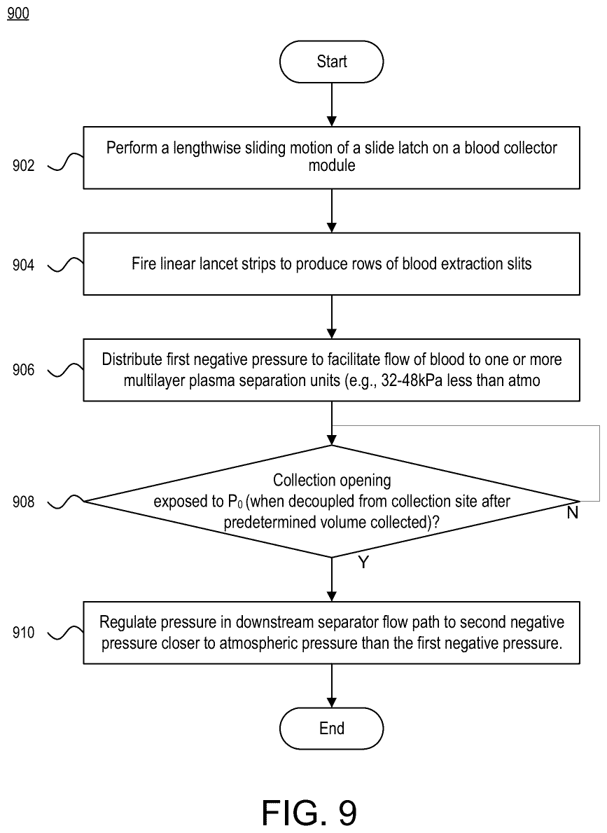

In some implementations, the blood collector module 102 includes a plasma separator module 106. In various examples, the plasma separator module 106b is integrally coupled to the blood collector module 102b as described with respect to FIGS. 4A and 4B. In certain examples, the plasma separator module 106a is separably coupled to the blood collector module 102a as described with respect to FIGS. 3H and 3I. In such implementations, the plasma separator module 106 includes one or more multilayer plasma separation units 310, 410 and a dynamic modular depressurization regulator 324, 424 as described with respect to FIGS. 3D-3H that perform dynamic modular pressure deregulation to facilitate separation of plasma from blood using a a plasma separation membrane in the one or more plasma separation units by regulating a downstream separator flow path 308 to second negative pressure 328. Detailed process steps for this aspect of the method 900 are described with respect to FIG. 9.

From a user perspective, the method 800 continues and includes decoupling 812 the blood collector module from the collection site after indication that blood collection is complete and the blood collector module coupled with the plasma separator module separate the plasma in liquid form using dynamic modular depressurization to accelerate the plasma flow through the plasma separation membrane with low risk of hemolysis and without additional user action.

In certain examples, the method 800 continues and includes transferring 814 blood components or plasma if plasma is collected (and in certain implementation may also include reagents) from an opening of the blood collector module to a transfer module 104. In one or more example implementations, the method 800 continues and includes performing 816 a biometric scan to confirm the identity of subject in a usage session that includes collection of the blood components from the subject.

In some examples, the process tracking module 108 is optionally configured to perform the biometric scan to confirm subject identity within a predetermined time period that also includes scanning the NFC tag using the same device used for performing the biometric scan. For example, because the blood collector module is coupled to the region of skin of the subject, a biometric scan such as a facial recognition scan can be performed within the same predetermined time period that the NFC tag is being scanned by the same device (e.g., a smart phone, a tablet, and the like). For blood tests where it is important to securing ensure the identity of the subject from who blood is being collected (e.g., drug test, medical testing, and so forth), the secure coupling to the shoulder or forearm of the subject is useful because either the front-facing camera, the back-facing camera, or fingerprint scanner, of the device may capture biometric measurements (e.g., facial, iris, or fingerprint scan” while within a distance near enough to perform the NFC scan of the blood collector module.

The method 800 continues and includes receiving 818 an indication 607 from the subject 116 indicating whether the blood collection was performed according to the communicated blood collection parameters which may be considered part of the reported results for the subject 116.

In various implementations, individual acts of the method 800 or combinations of acts of the method 800 for modular self-collecting of capillary blood may be performed using the system 100, the blood collector module 102, the plasma separator module 106, and/or the transfer modules 104 (such as for example, the sample tube 105, and/or a reagent mixer module 107).

As healthcare becomes increasingly personalized and remote, home-use or point-of-care blood collection devices such as the blood collector module 102a with the separably coupled plasma separator module 106a and the blood collector module 102b with an integrally coupled plasma separator module 106b. These devices not only allow for simple and efficient blood collection by the user, but the plasma separator modules 106a,106b efficiently separates plasma from the other blood components, which can then be sent to a laboratory for detailed analysis.

In some examples, a user may be instructed to send the plasma separator module 106 itself to the laboratory. Stoppers seals or the containers may be provided to seal the collector transfer port 244 or plasma output port 306 and the collection opening 235, 435 to better preserve the components during transport to the laboratory.

In such examples, residual blood components that remain on the plasma separation membrane 317,417 of each of the one or more multilayer plasma separation units 310,410 of the plasma separator module 106a, 106b can be extracted and analyzed. Residual blood component extraction can be an important differentiator for users, laboratories, and clinicians. Blood is a complex mixture of cells, platelets, and plasma. When blood is passed through the plasma separation membrane 317,417 in plasma separator module 106a,106b, cellular components (red and white blood cells) and platelets are typically retained by the membrane while plasma passes through. In various example implementations, such residual components can be extracted using one or more of the following processes.

One example of a process for extracting residual blood components from plasma separation membranes 317,417 is elution which involves passing a solvent (usually a buffer solution) through the plasma separation membrane 317,417 to effectively remove and collect residual blood components adhering to it. This buffer could be a saline solution, which maintains the integrity of the cells and platelets and allows them to be collected in a concentrated form. In some implementations gentle agitation or shaking of the one or more plasma separation units 310,410 in a buffer solution can help dislodge the residual components from the plasma separation membrane 317, 417. This should be done with care to avoid damaging the cells and platelets.

The inventors of the subject matter disclosed herein have determined that providing the ability to send residual blood components retained by plasma separation membranes may be of great importance for laboratory analysis. Some of the benefits may include: 1. Comprehensive Data Collection—by analyzing not just the plasma but also the residual components, a more complete picture of a patient's health can be obtained; 2. Cellular Analysis—residual components include red and white blood cells. Abnormalities in these cells can be indicative of various diseases including anemia, infection, and certain cancers. 3. Platelet Analysis—Platelets are vital for clotting. Abnormal platelet counts or morphology can indicate various health issues, including clotting disorders; 4. RNA/DNA Analysis—Extracting and analyzing residual components can allow for genetic analysis, which is increasingly used in personalized medicine approaches. 5. Cost and Time Efficiency—Sending the plasma separator module 106 along with the plasma 128 can minimize the need for a second blood draw, reducing patient discomfort and healthcare costs.

Accordingly, the inventors of the subject matter herein have determined that sending the plasma separator module 106 to the laboratory along with the plasma 128 has several potential advantages. It allows for the extraction and analysis of the residual blood components that are left on the plasma separation membrane after plasma extraction. These residual components can offer valuable information about a patient's health that is not apparent from plasma analysis alone. With careful extraction techniques, these components can be analyzed in a way that provides comprehensive, valuable data while minimizing patient discomfort and healthcare costs.

FIG. 9 is schematic flow chart diagram of a method 900 of performing plasma separation according to one or more examples of the disclosure. In various example implementations, the method 900 begins and includes in response to a user performing 902 a lengthwise sliding motion of a slide latch on a blood collector module sealingly coupled to a collection site on a body part of a subject, firing 904 a plurality of linearly-arranged lancet strips to produce rows of blood extraction slits and distributing 906 a first negative pressure within a plasma separator module coupled to the blood collector module to facilitate flow of blood from the collection site to an upstream entry surface of one or more multilayer plasma separation units.

In such implementations, the method 900 further includes, in response 908 to a collection opening 235,435 of the blood collector module 102a, 102b being decoupled from the collection site 120 after a predetermined volume of blood has been collected, regulating 910 pressure in a downstream separator flow path of the plasma separator module to a second negative pressure that is closer to current local atmospheric air pressure (P₀) than the first negative pressure to limit pressure exerted on the blood as it is separated by the plasma separation membrane of the one or more plasma separation units.

In various implementations, the first negative pressure is from about 32 kPa to about 48 kPa less than P₀ and the second negative pressure is from about 7 kPa to about 14 kPa less than P₀.

It may be noted that one or more of the actions or steps of the method 900 may be performed with different timing or in a different order depending on the configuration of the timing relative to the lengthwise sliding motion of the slide latch of the blood collector module as described with respect to FIGS. 2D-2H. For example, in certain limitations, the timing of distributing the first negative pressure may be varied relative to the firing of the lancet strips depending on the application and configuration of the blood collector module. Similarly, the timing of regulating the downstream separator flow path to a second negative pressure may vary depending on the volume of blood and/or plasma to be collected and the time to decouple the blood collector module from the collection site.

In certain examples, after the plasma is collected, the plasma separator module 106 may be discarded according to applicable guidelines. Beneficially, a single-use plasma separator module may be used effectively without concerns about buildup of blood cell components that reduce the effectiveness of the plasma separation membrane.

In one or more implementations, the method 900 may be performed by the apparatus 300 or the apparatus 400 and more specifically by the plasma separator module 106a coupled to the blood collector module 102a or the plasma separator module 106b (described with respect to FIGS. 3A-3I with specific modifications further described with respect to FIG. 4A-4B) integrally coupled to the blood collector module 102b described with respect to FIGS. 2A-2Q with specific modifications further described with respect to FIG. 4A-4B).

Clauses describing various implementations or embodiments of the present disclosure are provided below.

Clause 1. An apparatus comprising: a blood collector module comprising: a proximal collector portion comprising a dynamic modular depressurization chamber, a depressurization piston, a precompressed volume expansion spring, and a slide latch for actuation; a distal collector portion comprising a blood extraction chamber formed in a collector base and configured to collect blood and plasma therein a collection site in skin of a subject; a mid collector portion formed in the collector enclosure between the proximal collector portion and the distal collector portion of the collector enclosure to facilitate secure holding of the blood collector module between two digits of a hand, the mid collector portion further comprising a pressure distribution channel formed in the collector base and configured to distribute negative pressure generated by the dynamic modular depressurization chamber to the blood extraction chamber and to a transfer module coupled to the blood collector module; a sealing surface at a bottom portion of the blood collector module and is configured to stably seal the blood collector module to the skin around the collection site; a slide latch configured to be actuated by a lengthwise sliding motion of a single digit of the hand relative to the blood collector module; and a lancet carrier disposed within the distal collector portion and comprising one or more lancets wherein, in response to the lengthwise sliding motion of the slide latch, the lancet carrier fires the one or more lancets to momentarily puncture and retract from one or more blood extraction slits in the collection site; wherein, in response to the one or more blood extraction slits being produced, blood components are guided to flow from an opening in the sealing surface through a non-microfluidic blood flow channel for further processing.

Clause 2. The apparatus of clause 1, further comprising an angled collector transfer port disposed distally of the blood collector module and angled away from the subject at an acute angle of between 10 and 45 degrees relative to the sealing surface, the angled configured to transfer blood and/or blood components extracted by the blood collector module to the transfer module using the negative pressure generated by the dynamic modular depressurization chamber.

Clause 3. The apparatus of clause 2, wherein the lengthwise sliding motion of the slide latch is in a direction of blood and/or blood components flow from the collection site towards the angled collector transfer port.

Clause 4. The apparatus of clause 2, wherein the angled collector transfer port is configured to fluidically seal around an outside perimeter of the transfer module.

Clause 5. The apparatus of clause 1, wherein in response to the slide latch being actuated by the lengthwise sliding motion, an actuation indicator is configured to visually indicate that the blood collector module has been actuated.

Clause 6. The apparatus of clause 1, wherein in response to the slide latch being actuated by the lengthwise sliding motion, an actuation indicator is configured to haptically indicate that the blood collector module has been actuated.

Clause 7. The apparatus of clause 1, wherein in response to the slide latch being actuated by the lengthwise sliding motion, the precompressed volume expansion spring is released causing depressurization within the dynamic modular depressurization chamber to generate the negative pressure that is distributed by the pressure distribution channel to accelerate flow of blood and/or blood components.

Clause 8. The apparatus of clause 6, wherein the negative pressure in the blood extraction chamber causes one or more selected blood extraction slits to widen to enhance blood flow.

Clause 9. The apparatus of clause 1, wherein the one or more lancets comprise lancets wherein at least a piercing portion of the lancets comprise a single-bevel blade edge.

Clause 10. The apparatus of clause 1, wherein the one or more lancets comprise lancets strips that are aligned to cause a plurality of rows of blood extraction slits in the skin to be generally aligned with a blood flow direction from the blood collector module to the transfer module to inhibit flow interference from edges of the blood extraction slits.

Clause 11. The apparatus of clause 1, wherein the blood collector module is sealed to the skin around the collection site with the transfer module being coupled to the blood collector module lower than the collection site to facilitate self-venting of the non-microfluidic blood flow channel.

Clause 12. The apparatus of clause 1, wherein at least a portion of the one or more lancets and/or the non-microfluidic blood flow channel of the blood collector module comprise a surface one which one or more blood flow facilitation agents selected from a vasodilator and/or an anticoagulant are applied.

Clause 13. The apparatus of clause 2, wherein with the transfer module being coupled to the blood collector module and disposed perpendicularly to a support surface, the angled collector transfer port causes the opening in the sealing surface of the blood collector module to face at least partially upward to inhibit blood residue from spilling from the opening during decoupling of the transfer module from the angled collector transfer port.

Clause 14. The apparatus of clause 1, further comprising a blood flow initiator strip adhesively coupled to an interior surface of a blood flow channel wall defining the non-microfluidic blood flow channel, the blood flow initiator strip comprising an absorption layer that retains an initial volume of potentially hemolyzed blood from the collection site.

Clause 15. The apparatus of clause 14, wherein the blood flow initiator strip further comprises a hydrophilic wicking layer that extends at least partially into the transfer module and facilitates separation of blood drops into the transfer module.

Clause 16. The apparatus of clause 1, wherein a maximum length of the non-microfluidic blood flow channel is less than a minimum inner diameter of the transfer module.

Clause 17. The apparatus of clause 1, wherein an inner minimum cross-sectional area of the non-microfluidic blood flow channel in the blood collector module is at least 30% as large as the minimum inner cross-sectional area of the transfer module through which blood collected using the blood collector module is configured to flow.

Clause 18. The apparatus of clause 1, wherein an inner minimum cross-sectional area of the non-microfluidic blood flow channel is within a range of from about 0.2 cm2 to about 0.5 cm2.

Clause 19. The apparatus of clause 1, further comprising: a plasma separator module comprising: a blood input port that at least partially defines an upstream separator flow path and couples to the non-microfluidic blood flow channel of the blood collector module; a plasma output port that at least partially defines a downstream separator flow path and is configured to transfer plasma to a sample tube with a predetermined outside diameter; a separator body portion that links the blood input port and the plasma output port, the separator body portion channels the blood from the upstream separator flow path to an upstream entry surface of one or more multilayer plasma separation units, wherein the separator body portion further comprises the downstream separator flow path that fluidically couples a downstream exit surface of the one or more multilayer plasma separation units to the plasma output port for performing dynamic modular depressurization of the upstream separator flow path and the downstream separator flow path; and a dynamic modular depressurization regulator that, in response to exposure of the upstream separator flow path to atmospheric pressure after it has been depressurized to a first negative pressure, reduces risk of hemolysis as plasma is separated through the one or more multilayer plasma separation units by regulating depressurization of the downstream separator flow path to a second negative pressure that does not go more than a predetermined limit below atmospheric pressure.

Clause 20. An apparatus comprising a plasma separator module comprising: a blood input port that at least partially defines an upstream separator flow path and couples to a non-microfluidic blood flow channel of a blood collector module; a plasma output port that at least partially defines a downstream separator flow path and is configured to transfer plasma to a sample tube with a predetermined outside diameter; a separator body portion that links the blood input port and the plasma output port, the separator body portion channels the blood from the upstream separator flow path to an upstream entry surface of one or more multilayer plasma separation units, wherein the separator body portion further comprises the downstream separator flow path that fluidically couples a downstream exit surface of the one or more multilayer plasma separation units to the plasma output port for performing dynamic modular depressurization of the upstream separator flow path and the downstream separator flow path; and a dynamic modular depressurization regulator that, in response to exposure of the upstream separator flow path to atmospheric pressure after it has been depressurized to a first negative pressure, reduces risk of hemolysis as plasma is separated through the one or more multilayer plasma separation units by regulating depressurization of the downstream separator flow path to a second negative pressure that does not go more than a predetermined limit below atmospheric pressure.

Clause 21. The apparatus of clauses 19 or 20, wherein the one or more multilayer plasma separation units comprise: a blood compartment layer that holds blood coming from the upstream separator flow path; an upstream blood gasket layered adjacent to the blood compartment layer; a plasma separation membrane layered adjacent to the upstream membrane blood gasket; a downstream plasma gasket layered adjacent to the plasma separation membrane; and a drainage layer layered adjacent to the downstream plasma gasket wherein the drainage layer is configured to channel the plasma to a plasma output conduit.

Clause 22. The apparatus of clauses 19 or 20, wherein a geometry of an upstream blood gasket blocks a flow of blood in a downstream direction from contacting edges of a plasma separation membrane.

Clause 23. The apparatus of clauses 19 or 20, wherein the plasma separator module comprises a first multilayer plasma separation unit and a second multilayer plasma separation unit wherein: the upstream portions of the first and second multilayer plasma separation units are arranged proximate to each other and share a blood compartment layer; and the downstream portions of the first and second multilayer plasma separation units are arranged apart from each other and a plasma output conduit of the second multilayer plasma separation unit is extended to fluidically couple with the plasma output conduit of the first multilayer plasma separation unit.

Clause 24. The apparatus of clause 21, wherein the plasma separation membrane comprises an asymmetric microfiltration membrane comprising one or more of polyethylene terephthalate, polysulfone, polyethersulfone, polyimide, and combinations thereof.

Clause 25. The apparatus of clauses 21, wherein by using dynamic modular depressurization with the plasma separation membrane, a percentage of plasma collected within 5 minutes of for a blood sample of between 400 μl and 500 μl exceeds 70 percent of available plasma with less than 2% hemolysis.

Clause 26. The apparatus of clauses 19 or 20, wherein the predetermined outside diameter of the sample tube is about 10 mm or about 13 mm.

Clause 27. The apparatus of clauses 19 or 20, wherein the blood transfer channel is formed with an inverted u-shape to channel blood to narrow ends of the inverted u-shape where there is higher surface tension and a rounded top portion of the inverted u-shape allows air to efficiently return upstream as the blood below flows downstream.

Clause 28. The apparatus of clauses 19 or 20, wherein the plasma separator module is integrally coupled to a bottom portion of the blood collector module.

Clause 29. The apparatus of clause 28, wherein a perimeter geometry of a separator enclosure that encloses side portions of the plasma separator module is a perimeter geometry of the blood collector module.

Clause 30. The apparatus of clauses 19 or 20, wherein the plasma separator module is separably coupled to a distal portion of the blood collector module.

Clause 31. The apparatus of clauses 19 or 20, wherein the transfer module comprises: a prepackaged additive selected to mix with the blood to facilitate performance of one or more predetermined laboratory tests. An NFC tag coupled to an interior surface of the blood collector module; and a process tracking module implemented at least partially on a processor of a portable electronic device and configured to perform a biometric scan to confirm subject identity within a predetermined time period that also includes scanning the NFC tag using the portable electronic device.

Clause 32. A method comprising: firing one or more lancets to produce one or more blood extraction slits in response to a lengthwise sliding motion of a slide latch on a blood collector module sealingly coupled to a collection site on a body part of a subject; distributing a first negative pressure to facilitate a flow of blood from the collection site to an upstream entry surface of one or more multilayer plasma separation units of a plasma separator module; and regulating a downstream separator flow path of the plasma separator module in response to a decoupling of the blood collector module from the collection site to a second negative pressure that is closer to a current local atmospheric pressure (P₀) than the first negative pressure to limit force exerted on blood as it is separated by passing through the one or more plasma separation units.

Clause 33. The method of clause 32, enhancing blood flow volume from the collection site by using one or more lancets that have chemically-etched single-bevel blade edges.

Clause 34. The method of clauses 32 or 33, wherein: the first negative pressure is from about 32 kPa to about 48 kPa less than P₀; and the second negative pressure is from about 7 kPa to about 14 kPa less than P₀.

Clause 35. The method of clause 32, 33, or 34, further comprising performing one or more of: mixing one or more first reagents with the predetermined volume of blood using the first negative pressure to facilitate the mixing; and mixing one or more second reagents with the plasma using the second negative pressure to facilitate the mixing.

Examples and implementations may be practiced in other specific forms. The described examples are to be considered in all respects only as illustrative and not restrictive, unless otherwise clear from context. The scope of the invention is, therefore, indicated by the appended claims rather than by the foregoing description. All changes which come within the meaning and range of equivalency of the claims are to be embraced within their scope.

What is claimed is:

1. An apparatus comprising:
a blood collector module comprising:
a proximal collector portion comprising a dynamic modular depressurization chamber, a depressurization piston, a precompressed volume expansion spring, and a slide latch for actuation;
a distal collector portion comprising a blood extraction chamber formed in a collector base and configured to collect blood and plasma therein from a collection site in skin of a subject;
a mid collector portion formed in a collector enclosure between the proximal collector portion and the distal collector portion of the collector enclosure to facilitate secure holding of the blood collector module between two digits of a hand, the mid collector portion further comprising a pressure distribution channel formed in the collector base and configured to distribute negative pressure generated by the dynamic modular depressurization chamber to the blood extraction chamber and to a transfer module coupled to the blood collector module;
a sealing surface at a bottom portion of the blood collector module and is configured to stably seal the blood collector module to the skin around the collection site;
the slide latch configured to be actuated by a lengthwise sliding motion of a single digit of the hand relative to the blood collector module;
a lancet carrier disposed within the distal collector portion and comprising one or more linearly arranged lancets strips comprising from three to six lancets per strip wherein in response to the lengthwise sliding motion of the slide latch, the lancet carrier fires the one or more linearly arranged lancet strips to momentarily puncture and retract from one or more rows of blood extraction slits in the collection site, wherein, in response to the one or more rows of the blood extraction slits being produced, blood is guided to flow from an opening in the sealing surface through a non-microfluidic blood flow channel for further processing; and
an angled collector transfer port disposed distally of the blood collector module and configured to cause the transfer module when coupled to be angled away from the subject at an acute angle of between 10 and 45 degrees relative to the sealing surface.

2. The apparatus of claim 1, wherein the angled collector transfer port is configured to transfer blood and/or blood components extracted by the blood collector module to the transfer module, when coupled, using the negative pressure generated by the dynamic modular depressurization chamber.

3. The apparatus of claim 2, wherein the lengthwise sliding motion of the slide latch is in a direction of blood and/or blood components flow from the collection site towards the angled collector transfer port.

4. The apparatus of claim 1, wherein in response to the slide latch being actuated by the lengthwise sliding motion, the precompressed volume expansion spring is released causing depressurization within the dynamic modular depressurization chamber to generate the negative pressure that is distributed by the pressure distribution channel to accelerate flow of blood and/or blood components.

5. The apparatus of claim 4, wherein the negative pressure in the blood extraction chamber causes at least a portion of the one or more blood extraction slits to widen to enhance blood flow.

6. The apparatus of claim 1, wherein one or more lancets are configured as a plurality of linearly-arranged lancets strips that comprise lancets in a converging arrangement to produce rows of blood extraction slits with distal ends of the rows closer together than proximal ends of the rows, wherein at least a piercing portion of the lancets comprise a single-bevel blade edge.

7. The apparatus of claim 1, further comprising:
a plasma separator module comprising:
a blood input port that at least partially defines an upstream separator flow path and couples to the non-microfluidic blood flow channel of the blood collector module;
a plasma output port that at least partially defines a downstream separator flow path and is configured to transfer plasma to a sample tube with a predetermined outside diameter;
a separator body portion that links the blood input port and the plasma output port, the separator body portion channels blood from the upstream separator flow path to an upstream entry surface of one or more multilayer plasma separation units, wherein the separator body portion further comprises the downstream separator flow path that fluidically couples a downstream exit surface of the one or more multilayer plasma separation units to the plasma output port for performing dynamic modular depressurization of the upstream separator flow path and the downstream separator flow path; and
a dynamic modular depressurization regulator that, in response to exposure of the upstream separator flow path to a local atmospheric pressure (P₀) after it has been depressurized to a first negative pressure, reduces risk of hemolysis as plasma is separated through the one or more multilayer plasma separation units by regulating depressurization of the downstream separator flow path to a second negative pressure that does not go more than a predetermined limit below P₀.

8. An apparatus comprising:
a plasma separator module comprising:
a blood input port that at least partially defines an upstream separator flow path and is configured to couple to a transfer port of a blood collector module;
a plasma output port that at least partially defines a downstream separator flow path and is configured to transfer plasma to a sample tube with a predetermined outside diameter when the sample tube is coupled to the plasma output port;
a separator body portion that links the blood input port and the plasma output port, the separator body portion channels blood from the upstream separator flow path to an upstream entry surface of one or more multilayer plasma separation units, wherein the separator body portion further comprises the downstream separator flow path that fluidically couples a downstream exit surface of the one or more multilayer plasma separation units to the plasma output port,
wherein when the blood input port of the plasma separator module is coupled to the blood collector module and the sample tube is coupled to the plasma output port:
an actuation of the blood collector module begins a collection of plasma by causing blood to flow from a collection site on skin of a subject through the upstream separator flow path to the upstream entry surface of a plasma separation membrane of each of the one or more multilayer plasma separation units; and
the collection of the plasma is completed by the plasma separation module using the one or more multilayer plasma separation units to perform plasma separation on the blood collected by the blood collector module and outputting separated plasma to the sample tube when the blood collector module is decoupled from the collection site.

9. The apparatus of claim 8, wherein the one or more multilayer plasma separation units comprise two or more multilayer plasma separation units operating in parallel with parallel output conduits to deliver plasma separated in parallel to one or more sample tubes or transfer modules.

10. The apparatus of claim 8, wherein the one or more multilayer plasma separation units comprise four or more multilayer plasma separation units operating in parallel with parallel output conduits to deliver plasma separated in parallel to one or more sample tubes or transfer modules.

11. The apparatus of claim 8, wherein the plasma separation membrane:
is initially configured in a dry state to enable air to be drawn upstream through the plasma separation membrane to equalize a negative pressure between the upstream separator flow path and a downstream separator flowpath into which plasma is configured to flow after being separated by the plasma separation membrane; and
is subsequently saturated with blood from the blood collection module to inhibit free flow of air through the plasma separation membrane and enable a pressurization difference to exist between the upstream entry surface of the plasma separation membrane and the downstream exit surface of the plasma separation membrane,
wherein in response to an upstream separator flowpath of the plasma separator module being exposed to local atmospheric pressure P₀, the negative pressure existing at the downstream exit surface of the plasma separation membrane in a blood-saturated state assists in drawing the plasma through the plasma separation membrane.

12. The apparatus of claim 8, further comprising a dynamic modular depressurization regulator that in response to exposure of the upstream separator flow path to a local atmospheric pressure P₀ after it has been depressurized to a first negative pressure, reduces risk of hemolysis as plasma is separated through the one or more multilayer plasma separation units by regulating depressurization of the downstream separator flow path to a second negative pressure that is closer to P₀ than the first negative pressure to limit force exerted on blood as it is separated by passing through the one or more multilayer plasma separation units.

13. The apparatus of claim 8, wherein the plasma separator module separates 70 percent or greater of available plasma with less than 2% hemolysis.

14. An apparatus for a blood collection device to comprising:
a lancet strip assembly comprising a plurality of linearly-arranged lancet strips, each linearly-arranged lancet strip including from 3 to 10 evenly distributed flat lancets aligned to configured to produce multiple rows of blood extraction slits in a collection site in skin of a subject; and
one or more channels between the plurality of linearly-arranged lancet strips,
wherein in response to actuation, a lancet carrier propels the plurality of linearly-arranged lancet strips towards the collection site to produce the multiple rows of blood extraction slits in the skin of the subject.

15. The apparatus of claim 14, wherein the plurality of linearly-arranged lancet strips are configured in a parallel arrangement with the lancets strips and the one or more channels between the plurality of lancet strips arranged parallel to each other.

16. The apparatus of claim 14, wherein the plurality of linearly-arranged lancet strips are configured in a converging arrangement with distal ends of the rows of the plurality of linearly-arranged lancet strips being spaced closer together than proximal ends of the rows of the plurality of linearly-arranged lancet strips.

17. The apparatus of claim 14, where the plurality of linearly-arranged lancets strips comprise flat lancets wherein at least a piercing portion of the flat lancets comprise a single-bevel blade edge.

18. A method comprising:
firing one or more lancets to produce one or more blood extraction slits in response to a lengthwise sliding motion of a slide latch on a blood collector module sealingly coupled to a collection site on a body part of a subject;
distributing a first negative pressure to facilitate a flow of blood from the collection site to an upstream entry surface of one or more multilayer plasma separation units of a plasma separator module; and
in response to a collection opening of the blood collector module being exposed to a current local atmospheric pressure (P₀) after a decoupling of the blood collector module from the collection site, regulating a downstream separator flow path of the plasma separator module to a second negative pressure that is closer to P₀ than the first negative pressure to limit force exerted on blood as it is separated by passing through the one or more multilayer plasma separation units.

19. The method of claim 18, wherein:
the first negative pressure is from about 32 kilo Pascals (kPa) to about 48 kPa less than P₀; and
the second negative pressure is from about 7 kPa to about 14 kPa less than P₀.
